SEQUENCE LISTING (4 sequences)

SEQ ID NO: 1 - DNA, 34 bp, Artificial
  primer
attttatgagctccaccatcttttattttgcttt

SEQ ID NO: 2 - DNA, 35 bp, Artificial
  primer
taaataaggatccacacaaaatagataaaaaggag

SEQ ID NO: 3 - DNA, 33 bp, Artificial Sequence
  primer
attttatatcgatttgttaaaccagcatcacga

SEQ ID NO: 4 - DNA, 35 bp, Artificial Sequence
  primer
taaataactcgagaaaattaaatgtctggctggat

(19) United States
(12) Patent Application Publication
(10) Pub. No.: US 2007/0031927 A1
(43) Pub. Date: Feb. 8, 2007

(54) Biological system and assay for identifying inhibitors of tubulin glutamylases

(75) Inventors: Jacek Gaertig, Athens, GA (US); Dorota Wloga, Athens, GA (US); Krzysztof Rogowski, Montpellier Cedex (FR)

Correspondence Address: MUETING, RAASCH & GEBHARDT, P.A., P.O. BOX 581415, MINNEAPOLIS, MN, 55458, US

(73) Assignee: University of Georgia Research Foundation, Inc., Athens, GA (US)

(21) Appl. No.: 11/479,098

(22) Filed: Jun. 30, 2006

Related U.S. Application Data
(60) Provisional application No. 60/695,776, filed on Jun. 30, 2005.

(51) Int. Cl. C12P 21/06; C12N 9/22; C07H 21/04; C12N 1/10; C12N 9/48

(52) U.S. Cl. 435/69.1; 435/199; 435/320.1; 435/258.1; 536/23.2; 435/212

(57) ABSTRACT

Tetrahymena is used as a host cell in a biological assay for identification of inhibitors of tubulin glutamylases.

This application claims the benefit of U.S. Provisional Application Ser. No. 60/695,776, filed Jun. 30, 2005, which is incorporated herein by reference in its entirety.

This invention was made with government support under a grant from the National Science Foundation, Grant No. MBC-0235826. The U.S. Government has certain rights in this invention.

### BACKGROUND OF THE INVENTION

Microtubules are fibers made of α-tubulin and β-tubulin dimers. Microtubules form cytoplasmic networks and serve as frameworks of important organelles, including the mitotic spindle, centrioles, cilia and bundles inside neurites. The biogenesis of microtubules involves the synthesis of tubulin polypeptides, chaperonin-assisted folding and dimerization of α-tubulin and β-tubulin, transport to the sites of assembly, nucleation, polymerization, deposition of post-translational modifications (PTMs), and binding of diverse microtubule-associated proteins (MAPs).

During mitosis, the microtubules undergoes dramatic changes to transit from an interphase monopolar organization to the bipolar spindle. Thus, tubulins are a major target of anti-cancer drugs which act by disrupting the dynamic properties of MTs during mitosis and in some cases inducing apoptosis. However currently available widely used microtubule-targeting compounds (such as vinblastine or paclitaxel) suffer from a major limitation—they target microtubules indiscriminately. Paclitaxel, for example, a compound that hyperstabilizes microtubules and blocks cells in mitosis, is currently the most widely used drug to treat ovarian, breast, lung cancers and AIDS-related Kaposi's sarcoma (Ring et al. (2005) Cancer Treat Rev 31, 618-627; Cheung et al., (2005) Oncologist 10, 412-426). However, paclitaxel produces strong side effects by affecting non-mitotic microtubules, in particular in nerve cells (Hennenfent et al. (2006). Ann Oncol. 17, 735-749). Paclitaxel also affects the bone marrow leading to hematopoietic deficiencies in ˜90% of patients (Hagiwara et al. (2004) Breast Cancer 11, 82-85). Ideally, future anti-microtubule compounds should affect as few cell types as possible besides intended targets. Humans have several isotypes of α-tubulin and β-tubulin, some of which are expressed in a restricted fashion. However, tubulins are highly conserved and differ mainly in the small portion of their primary sequence near the C-terminal end (Luduena (1998) Int. Review Cytol. 178, 207-274). Thus, developing isotype-specific inhibitors for tubulin primary polypeptides is likely to be difficult.

Post-translational modifications of microtubules are ubiquitously present in eukaryotes and their physiological importance is increasingly well documented (Rosenbaum (2000) Current Biol. 10, R801-R803, Westermann et al. (2003) Nat Rev Mol Cell Biol 4, 938-947). The most studied post-translational modifications include acetylation of α-tubulin, detyrosination of α-tubulin, palmitoylation of α-tubulin, and phosphorylation, glutamylation, and glycylation of α-tubulin and β-tubulin.

Post-translational modifications are believed to function in regulating interactions of microtubules with MAPs (such as dynein and kinesin motors) (Rosenbaum (2000) Current Biol. 10, R801-R803), Westermann et al. (2003) Nat Rev Mol Cell Biol 4, 938-947). Most post-translational modifications are located on the C-terminal tails of tubulins, highly flexible acidic domains present on the surface of microtubules (Nogales et al. (1999) Cell 96, 79-88). The tails are also the major sites of interactions with kinesin and dynein motors, structural MAPs (MAP2, Tau), microtubule-severing protein katanin, and plus end-depolymerizer (MCAK) (Skiniotis et al. (2004) Embo J 23, 989-999, Ovechkina et al. (2002) J Cell Biol 159, 557-562, Lu et al. (2004) Mol Biol Cell 15, 142-150). By regulating the activity of tubulin modifying enzymes, cells can mark microtubules in specific subcellular areas to regulate binding and activity of MAPs in a localized fashion. Detyrosination of α-tubulin promotes transport of vimentin intermediate filaments mediated by kinesin-1 (Kreitzer et al. (1999) Mol. Biol. Cell 10, 1105-1118). Another post-translational modification, polyglycylation, appears to acts as a mark to regulate assembly of cilia and severing of stable cortical microtubules in Tetrahymena (Thazhath et al. (2002) Nature Cell Biol. 4, 256-259; Thazhath et al. (2004) Mol Biol Cell 15, 4136-4147). The basic principle of specific post-translational modifications acting alone or in combination to regulate binding of a variety of microtubule interactors is likely to be general. By analogy with the epigenetic “histone code”, eukaryotic cells appear to utilize a “microtubule code” to coordinate MAPs.

Glutamylation, a conserved post-translational modification, occurs by addition of a variable number of glutamate (also known as glu or “E”) residues onto specific glutamate residues of the C-terminal tail domain of α-tubulin or β-tubulin (Edde et al. (1990) Science 247, 83-85), Weber et al. (1996) FEBS Lett. 393, 27-30). Besides tubulins only the nucleosome assembly proteins, NAP-1 and NAP-2 are known to undergo glutamylation (Regnard et al. (2000) J. Biol. Chem. 275, 15969-15976). The added glutamates form a peptide side chain using the gamma-carboxyl group of the glutamate in the primary sequence. Due to its negative charge and bulky nature, glutamate side chains have a strong structural impact on microtubules. Because the number of added glutamates changes as the function of age of microtubules and cell cycle stage, this post-translational modification is probably not a simple on/off signal. Rather, glutamylation may act like a rheostat to fine tune the function of microtubules. Tubulin glutamylation is particularly abundant on microtubules inside cellular projections including neuritis (Wolff et al. (1992) Eur. J. Cell Biol. 59, 425-432) and cilia (Bré et al. (1994) Cell Motility and the Cytoskeleton 27, 337-349). Glutamylation accumulates on microtubules of centrioles and in the central part of the mitotic spindle (Bobinnec et al. (1998) Cell Motil. Cytoskeleton 39, 223-232). Tubulin glutamylation is important in vivo. Injection of antibodies specific to glutamylated tubulin caused disassembly of centrioles in mammalian cells (Bobinnec et al. (1998) J. Cell Biol. 143, 1575-1589), suggesting that inhibitors of glutamylation could have anti-mitotic properties. A mutation of a subunit associated with the TTLL1 glutamylase in the mouse blocked assembly of sperm axonemes and affected behavior (Campbell et al. (2002) Genetics 162, 307-320). In vitro, glutamylation of microtubules strongly affects binding of kinesins and structural MAPs, MAP2 and Tau (Bonnet et al. (2000) J. Biol. Chem. 276, 12839-12848, Boucher et al. (1994) Biochemistry 33, 12471-12477).

Inhibitors of aforward post-translational modification enzyme are not known in the art. Furthermore, inhibitors are known for only one of the reverse post-translational modification enzymes, tubulin deacetylase HDAC6 (related to histone deacetylases). Overexpression of HDAC6 decreased acetylation and increased chemotactic motility of mammalian cells (Hubbert et al. (2002) Nature 417, 455-458). HDAC6 can be inhibited with trichostatin A (a broad inhibitor of deacetylases) (Matsuyama et al. (2002) Embo J 21, 6820-6831), and tubacin (a specific inhibitor) (Haggarty et al. (2003) Proc. Natl. Acad. Sci. U.S.A. 100, 4389-4394). Chemically blocking HDAC6 increased the level of acetylation on microtubules and decreased cell motility as well as disrupted localization of the p58 MAP (a Golgi-microtubule linker) in vivo (Haggarty et al. (2003) Proc. Natl. Acad. Sci. U.S.A. 100, 4389-4394). Inhibitors of HDAC6 has helped to uncover potential new functions for α-tubulin acetylation, including its role in the immune synapse formation (Serrador et al. (2004) Immunity 20, 417-428), and during infection of cells by HIV (Valenzuela-Femandez et al. (2005) Mol Biol Cell 16, 5445-5454). HDAC6 is upregulated in the acute myeloid leukemia cells (Bradbury et al. (2005) Leukemia 19, 1751-1759), and is one of the estrogen-responsive genes in breast carcinoma. Blocking HDAC6 with tubacin inhibited estradiol-induced cell migration of breast carcinoma cells (Saji et al. (2005) Oncogene 24, 4531-4539). Tubacin also increased anti-cancer effects of other compounds, including a proteasome inhibitor, bortezomid (Hideshima et al. (2005) Proc Natl Acad Sci U S A 102, 8567-8572). Although HDAC6 effects could be mediated by at least two different substrates (α-tubulin and HSP90, (Kovacs et al. (2005) Mol Cell 18, 601-607), it is very likely that the effects on cell motility are microtubule-mediated. Importantly, HDAC6 is required for the synergistic inhibitory action of paclitaxel and lonafarmib (an inhibitor of famesyltransferase) on cancer cells (Marcus et al. (2005). Cancer Res 65, 3883-3893). However, these targeting efforts are limited to one post-translational modification and specifically, to one enzyme, HDAC6 deacetylase. A general strategy for identifying post-translational modification drugs and in particular inhibitors of forward enzymes responsible for deposition of PTMs is needed.

### SUMMARY OF THE INVENTION

The invention provides a biological system and assay for identification of inhibitors of enzymes responsible tubulin glutamylation.

In one aspect, the invention is directed to a biological system that includes a host protozoan, preferably a ciliate such as Tetrahymena, that overexpresses a tubulin glutamylase, preferably Tetrahymena Ttll6Ap, Ttll6Bp, Ttll6Cp, Ttll6Dp, Ttll6Ep or Ttll6Fp. In one embodiment, the Tetrahymena overexpresses a wild-type tubulin glutamylase. In another embodiment, the Tetrahymena expresses a modified tubulin glutamylase, for example a truncated form of a Tetrahymena tubulin glutamylase that is missing the C-terminal region responsible for targeting the glutamylase to the cilia. A modified tubulin glutamylase can include, for example, a subunit of a tubulin glutamylase or a mutated tubulin glutamylase having mutations at one or more sites, or a fusion construct that includes all or part of a tubulin glutamylase. A preferred truncated tubulin glutamylase is Tetrahymena Ttll6Ap-Δ₇₁₀. Preferably, the tubulin glutamylase is under the control of a metallothionein promoter, more preferably an MTT1 promoter. MTT1 is conveniently inducible by cadmium.

The invention should be understood as including a Tetrahymena cell or cell line that overexpresses a wild-type or modified tubulin glutamylase as described above. In a particularly preferred embodiment, the Tetrahymena cell or cell line contains polynucleotide sequence encoding a wild-type or modified tubulin glutamylase that is integrated into the BTU1 locus. A preferred construct for use in transforming the Tetrahymena includes an MTT1 promoter region upstream of a region that encodes a fusion protein. The fusion protein optionally includes a marker protein, such as green fluorescent protein (GFP), followed by a tubulin glutamylase, or vice versa. Preferably, the construct encodes the marker protein followed by an in-frame coding region of a genomic sequence of the tubulin glutamylase gene, preferably the Tetrahymena TTLL6A gene (locus number 25.m00404 at Tetrahymena genome database; The Institute for Genomic Research website at http://www.tigr.org), or a truncated version thereof, TTLL6A-Δ₇₁₀. A particularly preferred Tetrahymena is one that expresses Ttll6Ap-GFP or Ttll6Ap-Δ₇₁₀-GFP.

In another aspect, the invention is directed to a Tetrahymena tubulin glutamylase, as well as a nucleic acid encoding a Tetrahymena tubulin glutamylase. The invention encompasses amino acid sequences having at least about 90%, preferably at least about 95%, and more preferably at least about 98% identity to the amino acid sequences described herein.

In another aspect, the invention is directed toward a high throughput in vivo assay for identification, validation and/or analysis of selective inhibitors of tubulin glutamylases. The assay is performed using a protozoan, preferably the ciliate Tetrahymena, a model protist with a sophisticated microtubular cytoskeleton. The Tetrahymena used in the in vivo assay overexpresses a tubulin glutamylase, as described herein. This assay is a phenotypic screen based on rescue from growth arrest caused by overexpression of a tubulin glutamylase enzyme in Tetrahymena.

In another aspect, the invention is directed toward an in vitro assay for identification, validation and/or analysis of selective inhibitors of tubulin glutamylases. This assay makes use of a glutamylation reaction using a purified Tetrahymena enzyme and microtubules. A variant of this assay, useful as a primary screen, employs luciferase activity as a readout signal.

Inhibitors of tubulin glutamylases are expected to be therapeutically useful to treat or prevent many diseases including cancer and disorders of central nervous system including mental diseases. They may also find utility as a male contraceptive.

Accordingly, the present invention is directed to a Tetrahymena cell that overexpresses a tubulin glutamylase. Preferably the Tetrahymena includes a polynucleotide operably encoding a tubulin glutamylase, wherein the polynucleotide is integrated into a BTU1 locus. The polynucleotide preferably includes an MTT1 promoter region upstream of a region encoding a fusion protein, which fusion protein incorporates a marker protein and a tubulin glutamylase. The marker protein is preferably an optically detectable protein; more preferably it is a fluorescent protein such as green, red or blue fluorescent protein. The tubulin glutamylase may be a wild-type or a modified tubulin glutamylase; preferably the tubulin glutamylase is Tetrahymena Ttll6Ap glutamylase or Ttll6Ap-Δ₇₁₀ glutamylase. A preferred Tetrahymena cell is one that overexpresses Tetrahymena TTLL6A gene (locus number 25.m00404 at Tetrahymena genome database), or its truncated version, Tetrahymena TTLL6A-Δ₇₁₀.

The invention further provides a method for identifying an inhibitor of tubulin glutamylase that includes contacting a Tetrahymena cell of the invention with a candidate inhibitor compound; and detecting an increase in cell growth and/or motility; wherein an increase in cell growth or motility is indicative of tubulin glutamylase inhibition.

Unless otherwise specified, “a,” “an,” “the,” and “at least one” are used interchangeably and mean one or more than one.

### BRIEF DESCRIPTION OF THE DRAWINGS

The patent or application file contains at least one drawing executed in color. Copies of this patent or patent application with color drawings will be provided by the Office upon request and payment of the necessary fee.

FIG. 1 shows ciliary localization of Ttll6Ap-GFP (left panel, A), immuno-fluorescence with the anti-glutamylation antibody of cells overproducing Ttll6Ap (middle panel, E) and controls (right panel, H). Abbreviations: sc, subcortical MTs; ic, intracytoplasmic MTs; oc, oral cilia; oa, oral apparatus.

FIG. 2 shows that hyperglutamylation by Ttll6Ap-ΔCT-GFP stabilizes cytoplasmic MTs. Cells overproducing truncated Ttll6Ap-GFP or control cells were treated with 40 uM nocodazole for 30 minutes and labeled by immunofluorescence using anti-α-tubulin antibodies. Note the presence of drug-stable wavy and thick MTs in the cell body of Ttll6Ap-ΔCT cells.

FIG. 3 shows exemplary in vivo and in vitro screens for inhibitors of glutamylases.

FIG. 4 shows fluorescence intensity as a function of Tetrahymena cell concentration; cells with MTT1-driven GFP, 100 μl/well, 96 well black plastic optical bottom plates, 485/530 nm.

FIG. 5 shows characterization of the neuronal glutamylase. (A) A Coomassie Brilliant Blue stained NEPHGE 2D gel of a purified glutamylase fraction, immunoprecipitated from 200 3-day old mouse brains using mAb 206. All numbered protein spots were identified by nano-LC-MS-MS (Table I). “HC” and “LC” mark the positions of heavy and light chains of mAb 206. (B) Immunoprecipitations with mAb 206, L83 (anti-p32/PGs1; Regnard et al., J Cell Sci 116, 4181 (2003)), L80 (anti-p79) and DM 1A (Mab anti-α-tubulin) used as a control. Equal proportions of input and supernatants were assayed for glutamylase activity. (C) Input, supernatants and beads (equal proportions) were analyzed by western blotting. The glutamylase activity and all 5 glutamylase complex subunits were strongly depleted from the supernatants. The proteins were quantitatively recovered with the beads of mAb 206, L83 and L80, but not of DM1A. Arp1 did not quantitatively co-purify with the enzyme. Janke et al. (2005) Science 308, 1758-1762.

FIG. 6 shows a structural model of TTLL1. (A) Crystal structure of glutathione synthetase from E. coli (pdb code 1GSA) in a complex with ADP, glutathione, Mg²⁺ and sulphate (Hara et al., Biochemistry 35, 11967 (1996)). (B) A model of mouse TTLL1 (PGs3) in a complex with ATP, Mg²⁺ and a protein substrate shown in ball-and-stick representation. The regions of TTLL1 that could not be modeled are drawn as thin lines. (C) A close-up view of the active center of TTLL1. The protein substrate is a three-residue peptide with a central modified glutamate (backbone in medium green). The flanking amino acids are drawn only with Cβ atoms for clarity of the picture. The glutamate side chain contains two additional glutamate residues (Glu 1, Glu 2). The putative site for the next glutamate residue to be added to the side chain is indicated (Glu 3 site). All residues of the active site that are conserved with other ATP-dependent carboxylate-amine ligases are shown in dark green (see FIG. S2). Some positively charged amino acid residues (K13, K142 and K215 in light grey) that are specific to TTLL1 and close to the active site might be important for substrate binding. The proximity of the carboxyl group of Glu 2 and the phosphate of ATP could catalyze the formation of an acylphosphate intermediate (broken line). Oxygen, nitrogen, phosphate, magnesium atoms are in red, blue, magenta and orange, respectively. Janke et al. (2005) Science 308, 1758-1762.

FIG. 7 shows that Ttll1p of Tetrahymena is associated with tubulin glutamylation. (A) A cell expressing Ttll1p-GFP, labeled by immunofluorescence using anti-GFP antibodies. (B-D) Glutamylated MTs labeled by ID5 antibody in a cell overproducing Ttll1p-GFP (B), a wild type cell (C) and a TTLL1-null cell (D). Note a strong reduction in the labeling of rows of basal bodies in D as compared to C. (E) Immunoblotting studies on total Tetrahymena cell extracts before (−) and after (+) 3 hrs cadmium induction of Ttll1p-GFP, analyzed with anti-GFP (GFP), anti-glutamylation GT335 and polyE antibodies (arrowhead points to Ttll1p-GFP; * marks the comigrating α- and β-tubulin bands). (F) The soluble (S) and cytoskeletal (C) fractions from cells overproducing Ttll1p-GFP (+) or non-induced controls (−) were analyzed for tubulin glutamylase activity in vitro. (G, H) Soluble fractions of cells overproducing Ttll1p-GFP were subjected to immunoprecipitation with anti-GFP antibodies. The input (In), unbound (Un) and bound (Bo) fractions were analyzed by immunoblotting with anti-GFP antibodies (G; arrowhead TG points to Ttll1p-GFP, HC to the anti-GFP heavy chain) and assayed for glutamylase activity in vitro (H). Equal volumes of input, unbound and bead fractions were analyzed by immunoblotting, while 20 times more of bead fraction was assayed for glutamylase activity. Abbreviations: oa, oral apparatus; noa, new oral apparatus prior to cell division; df, deep fiber; bb, basal bodies. Janke et al. (2005) Science 308, 1758-1762.

FIG. 8 shows an evolutionary tree of TTL domain proteins, based on the Neighbor-Joining method. Numbers correspond to bootstrap values for 100 repeats. Colored dots represent predicted TTL domain sequences in several genomes analyzed. The grey lines denote clades discussed in the paper. The yeast sequence was used as an outgroup. See FIGS. S3, S4 for complete data. Janke et al. (2005) Science 308, 1758-1762.

FIG. 9 shows that Ttll6p-GFP is associated with strong tubulin glutamylation in vivo and in vitro. (A-D) GFP fluorescence (A, B) and immunofluorescence images using ID5 anti-glutamylation antibody (C, D) of cells overproducing Ttll6Ap-GFP (A, C) or Ttll6ApΔ₇₁₀-GFP (B, D). The staining detected in A, B co-localized with MTs visualized by anti-α-tubulin antibodies (results not shown). Note appearance of strong glutamylation in the cell body in cells overproducing forms of Ttll6Ap, while glutamylation is restricted to cilia and basal bodies in control cells (compare with the wild type cell shown in FIG. 3C). Abbreviations: c, cilia; oc, oral cilia; oa, oral apparatus; bb, basal bodies; sc, subcortical MTs, ic, intracytoplasmic MTs. (E) Tubulin glutamylase assays using soluble (S) and cytoskeletal (C) fractions from Ttll6ApΔ₇₁₀-GFP cells grown in the absence (−) and presence (+) of cadmium for 3 hrs. (F) Immunoblots of total cells (anti-GFP) or cytoskeletons of the following strains following a 3 hr cadmium treatment: wild type (WT), cells overproducing GFP (GFP), Ttll6ApΔ₇₁₀-E422G-GFP (Δ710-G), Ttll6ApΔ₇₁₀-GFP (Δ710), and Ttll6Ap-GFP (Ttll6p). The antibodies used were: anti-GFP (GFP), anti-α-tubulin (12G10), anti-glutamylation (ID5), anti-polyglycylation (polyG). (G-I) Cells overproducing an enzymatically active Ttll6Ap and not an ATPase-deficient form fail to multiply and undergo ciliary paralysis. Growth curves on SPP medium for strains expressing Ttll6Ap-GFP (G), Ttll6ApΔ₇₁₀-GFP or Ttll6ApΔ₇₁₀-E422G-GFP (H) with or without cadmium induction. Note that only the ATPase-capable proteins reduce the growth rate. (I) The percentage of motile cells in several overproducing strains. After 3 hrs of cadmium induction 100-200 cells were scored for vigorous motility. Either extremely sluggish (showing rotations but no directional movement) or completely paralyzed cells were counted as non-motile. The data shown in all panels represent mean values from three independent experiments. Janke et al. (2005) Science 308, 1758-1762.

FIG. 10 shows co-purification of the glutamylase subunits and enzymatic activity. Janke et al. (2005) Science 308, 1758-1762; online supporting material available on the worldwide web at sciencemag.org/cgi/content/full/1113010/DC1.

FIG. 11 shows a sequence profile alignment for TTLL1 modeling. Janke et al. (2005) Science 308, 1758-1762; online supporting material available on the worldwide web at sciencemag.org/cgi/content/full/1113010/DC1.

FIG. 12 shows a phylogenetic analysis of predicted protein gene sequences containing a TTL domain. Janke et al. (2005) Science 308, 1758-1762; online supporting material available on the worldwide web at sciencemag.org/cgi/content/full/1113010/DC1.

FIG. 13 shows a multiple sequence alignment of TTL domains of TTL and TTLL proteins. Janke et al. (2005) Science 308, 1758-1762; online supporting material available on the worldwide web at sciencemag.org/cgi/content/full/1113010/DC1.

FIG. 14 shows glutamylation activity of Ttll6Ap in vitro. Janke et al. (2005) Science 308, 1758-1762; online supporting material available on the worldwide web at sciencemag.org/cgi/content/full/1113010/DC1.

### DETAILED DESCRIPTION OF ILLUSTRATIVE EMBODIMENTS

The present invention is directed to a new group of enzymes which regulate microtubules, called tubulin glutamylases. The invention is further directed to polynucleotides encoding such enzymes as well as the polypeptide enzymes, including enzymatically active subunits, variants and analogs thereof. The enzymes are responsible for post-translational glutamylation of tubulin proteins in which a side branch made of one or more glutamic acid is added to the protein.

In the following examples, overproduction of one of these enzymes in Tetrahymena, Ttll6Ap, using MTT1 promoter is shown to lead to cell multiplication arrest of transgenic Tetrahymena. Advantageously, the lethal condition brought about by overproduction of the enzyme can be used to screen for novel compounds which inhibit the enzyme, based on a rescue of the lethal phenotype associated with overproduction. Thus, the invention includes screening methods to identify inhibitors of a tubulin glutamylase, as well as compounds identified using the screening method and methods of using them.

Tetrahymena as a Model System

Tetrahymena is a free-living ciliate and a model eukaryote with a sequenced genome. Tetrahymena has been used in research that led to some key achievements, including the Nobel award winning discovery of self-splicing RNA, and identification of telomeres and telomerase, histone acetyltransferase, dynein, and siRNAs that guide DNA rearrangement (Turkewitz et al. (2002) Trends in Genetics 18, 35-40). The Tetrahymena system is well equipped for reverse genetics (by homologous DNA recombination), and biochemical studies (Collins et al. (2005) Curr Biol 15, R317-318).

Tetrahymena assembles a diverse set of microtubules including those forming the cell body networks, cilia, centriole-like basal bodies, and the mitotic spindle. Due to the importance of their diverse microtubules, ciliates are sensitive to perturbations in microtubule-dependent functions which result in characteristic (potentially screenable) phenotypes (Janke et al. (2005) Science 308, 1758-1762 (see Example I), Thazhath, et al. (2002) Nature Cell Biol. 4, 256-259, Fujiu et al. (2000) Cell Motil Cytoskeleton 46, 17-27).

Tetrahymena posttranslationally modifies its microtubules, using highly conserved post-translational modifications, including extensive glutamylation of microtubules in the basal bodies and cilia (Gaertig (2000) Microbiol. 47, 185-190). Tetrahymena has been in use as a model to dissect the function of post-translational modifications. The use of Tetrahymena led to the first report a mutant phenotype caused by lack of specific sites of post-translational modifications on tubulins (Thazhath et al. (2002) Nature Cell Biol. 4, 256-259), and the discovery of the first post-translational modification forward enzyme (Janke et al. (2005) Science 308, 1758-1762 (see Example I)).

Tetrahymena is attractive for high throughput manipulations due to its: 1) rapid growth (generation time of 3 hrs), 2) low cost of culture, 3) nearly transparent culture medium, 4) ability to grow on defined medium without animal products (lowers the cost of culture and reduced the strigency of required biosafety procedures, 5) lack of pathogenicity, 6) routine culture on 96-well plates, 7) growth to high density in microdrops (compatibility with 384-1536 well plates), 8) lack of autofluorescence (no cell wall or plastids), 9) rapid cell motility (promotes mixing of assay components), 10) sensitivity to established inhibitors (e.g. cycloheximide, paclitaxel) within the concentration range similar to animal cells, 11) methods for introduction of transgenes and protein tagging, 12) targeted mutation approaches that allow for exploration of loss-of-function phenotypes, 13) inducible-repressible promoters that allow for generation of gain-of-function phenotypes, 14) large cell size -highly amenable for HT cytological profiling at low microscopic magnification, 15) advanced cellular functions shared with animal cells including sophisticated microtubule-based organelles, regulated secretion, nuclear apoptosis, DNA rearrangements, phagocytosis, and chemotactic cell motility.

Tubulin Glutamylases

Until now the enzymes which deposit post-translational modifications on microtubules remained unidentified due to, among other things, difficulties in their purification. As a result of synergistic research that explored advantages of the mouse model (in collaboration with Dr. Bernard Eddé and Carsten Janke at CRBM, Montpelier, France) and Tetrahymena, our laboratories identified a family of glutamylases differing in substrate preferences (α-tubulin vs. β-tubulin) (Janke et al. (2005) Science 308, 1758-1762 (see Example I)).

The catalytic subunit of the major neural glutamylase is the product of the TTLL1 gene that encodes a protein with a tubulin-tyrosine ligase (TTL)-like domain. TTL is a well known reverse enzyme for another post-translational modification, tubulin detyrosination (Ersfeld et al. (1993) J. Cell Biol. 120, 725-732). TTLL1 and TTL belong to a larger family of conserved proteins with a common catalytic domain. We named these proteins “TTL-like” (TTLLs). Phylogenetic analyses identified conserved subtypes of TTLLs, that may all be involved in ligation of specific amino acids to tubulins (Janke et al. (2005) Science 308, 1758-1762 (see Example I)). Members of this novel family are structurally related to other proteins designated as ADP-forming enzymes, that display an ATP hydrolysis-dependent carboxylate-amine ligase activity. TTLL1 type proteins are associated with glutamylase activity on α-tubulin. TTLL1-mediated glutamylation is required for neurite extension in the mouse (Janke et al. (2005) Science 308, 1758-1762 (see Example I)). A mutation in the noncatalytic subunit of TTLL1 complex, PGs1, led to disruption of assembly of axonemal microtubules in murine sperm and interestingly, caused reduction in aggressive behavior in males (Campbell et al. (2002) Genetics 162, 307-320). In Tetrahymena, a gene knockout of Ttll1p reduced the level of α-tubulin glutamylation, which in turn was associated with slow growth, defects in specific types of microtubules including basal bodies (centriole-like structures that template cilia), and slow ciliary beating (Janke et al. (2005) Science 308, 1758-1762 (see Example I)).

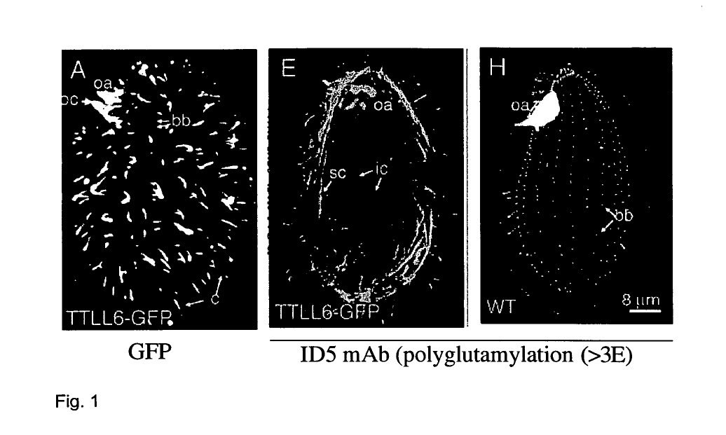

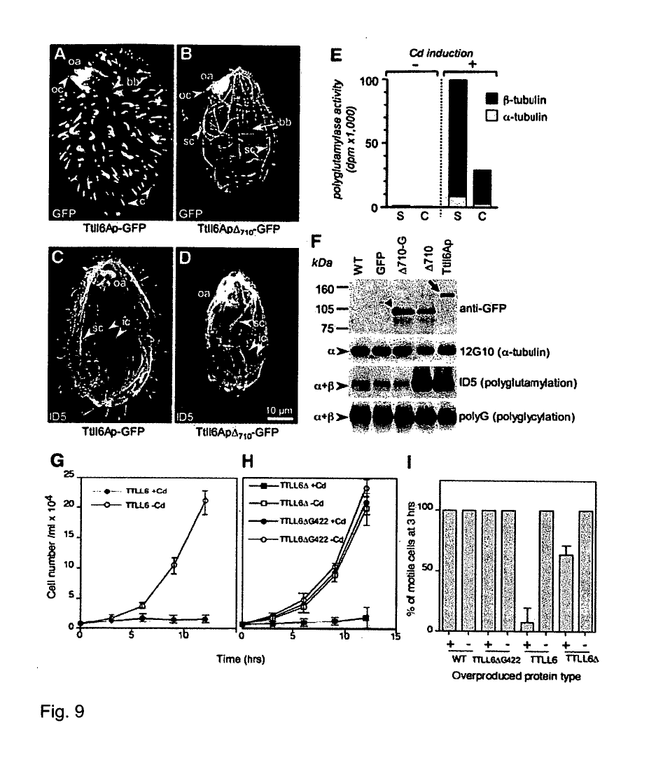

Another TTLL, TTLL6, was found to be a highly active glutamylase for β-tubulin (Janke et al. (2005) Science 308, 1758-1762 (see Example I)). A TTLL6 type protein of Tetrahymena, Ttll6Ap, localizes mainly to cilia. Tetrahymena cells overproducing Ttll6Ap-GFP showed a dramatic increase in the level of tubulin glutamylation on cilia and some cell body microtubules (compare FIG. 1, middle and right panels, with FIG. 9C). A reliable in vitro assay for glutamylation that includes tritiated glutamic acid, taxol-stabilized microtubules and ATP is available (Regnard et al. (1998) Biochemistry 37, 8395-8404, Regnard et al. (1999) J. Cell Sci 112, 4281-4289). Remarkably, crude extracts of Tetrahymena cells overproducing Ttll6Ap under a cadmium-inducible promoter had 100 times more of activity in vitro (as compared to non-induced cells) (Janke et al. (2005) Science 308, 1758-1762 (see Example I)). Ttll6Ap-GFP with a mutation in the predicted ATP-binding site lacked any detectable glutamylase activity. Thus, Ttll6Ap expresses well in Tetrahymena and large quantities of this enzyme can be produced cheaply.

Because there are several expressed paralogs, it has not yet been possible to determine what the loss of function for the Ttll6Ap-type activity is in Tetrahymena. A single knockout of TTLL6A gene did not produce a mutant phenotype. Hyperglutamylation of β-tubulin by Ttll6Ap stabilizes microtubules, blocks cells proliferation and inhibits ciliary motility in Tetrahymena.

A full-length Ttll6Ap localizes primarily to cilia in Tetrahymena (FIG. 1, left panel; Example I and FIG. 9A). When the full length Ttll6Ap-GFP was overproduced, cells stopped dividing in a few hours (Example I) and become paralyzed due to lack of ciliary beating (Example I).

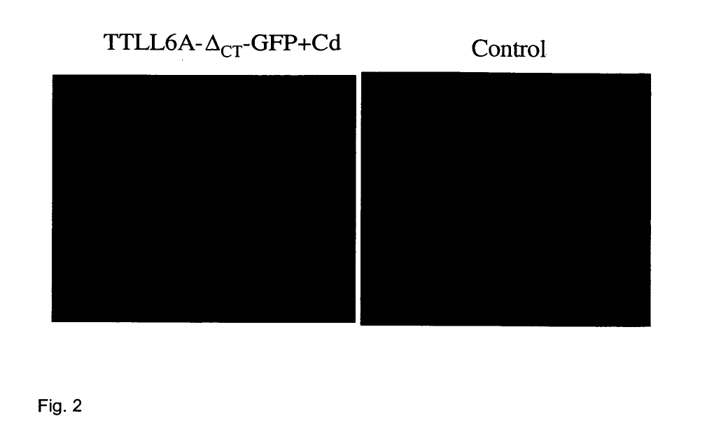

A truncated but enzymatically active form of Ttll6Ap lacking 288 amino acids of its C-terminus (Ttll6Ap-Δ₇₁₀) failed to localize to cilia and accumulated in the cell body. As Ttll6Ap-Δ710 accumulated on cell body microtubules, their appearance changed dramatically—they acquired extensive glutamylation and become thick and wavy, which indicated that these microtubules were hyperstable. Indeed we found that these microtubules were resistant to nocodazole (FIG. 2).

An inactive (ATPase-dead) variant of the truncated protein (Ttll6Ap-Δ₇₁₀-G422) accumulated to the same level as the enzymatically active protein, but failed to hyperglutamylate and hyperstabilize cell body microtubules. The ATPase-dead variant did not have any effect on cell growth and motility (Example I). This experiment indicates that the ATP-binding site within the TTL homology domain of the glutamylases is one of the preferred target region for development of specific inhibitors.

The above observations argue that the observed phenotypic effects of active Ttll6tAp are mediated by its glutamylation activity and not by the mere binding to microtubules. Importantly, excessive glutamylation on β-tubulin strongly inhibits cell multiplication and ciliary motility. The biological assay of the invention takes advantage of these effects, because a glutamylase inhibitor will rescue the host organism from a lethal condition, restoring growth and, where a full length glutamylase is used such that it localizes to the cilia, cell motility. Of course, glutamylation on b-tubulin may be required for cell viability. Therefore, the inhibitor would need to be applied in a range of concentrations to find its concentration that titrates out the overproduced enzyme but does not deplete the total activity below the endogenous level. It should be noted that cells overproducing Ttll6Ap-Δ_{CT} also ceased dividing, but the effect on ciliary motility was relatively mild because this isoform cannot be targeted to cilia (Example I). This is advantageous because the truncated Ttll6Ap variant is especially useful in the cell-based, biological assay to avoid sedimentation of paralyzed cells to the well bottom that could disturb the uniformity of signal strength across the well.

Antibody-Based Screening Assays

The invention further includes screening assays based on anti-glutamylation antibodies, which give an extremely strong signal reflecting Ttll6Ap activity (see Example I; FIG. 9C,D). Examples of antibody-based assays include cytoblot for an in vivo assay, or ELISA-type assay with plates coated with microtubules for in vitro assay.

Scientific and Medical Applications

It is expected that an inhibitor of glutamylase will be of great value in assessing the function of glutamylation in diverse cell types, including those that are difficult to grow in vitro or manipulate genetically and are known to have highly glutamylated microtubules (such as ciliated epithelial cells (Million et al. (1999)). There is evidence that glutamylation affects assembly of certain organelles such as flagella (Campbell et al. (2002) Genetics 162, 307-320), centrioles (Bobinnec et al. (1998) J. Cell Biol. 143, 1575-1589), and neural bundles (Janke et al. (2005) Science 308, 1758-1762 (see Example I)). Thus, genetic means alone may not adequately assess the function of glutamylation in already assembled organelles.

Additionally, glutamylase inhibitors could find innovative uses in medicine. As noted earlier, tubulins are a major target of anti-cancer drugs. However, tubulins are highly conserved. Thus, developing isotype-specific inhibitors for tubulin primary polypeptides is likely to be difficult. The present invention provides an alternative strategy that involves targeting the post-translational modification enzymes rather then tubulins per se. Tubulin glutamylases identified herein are suitable objects for highly focused targeting strategies. Mammals have nine glutamylase enzymes (Janke et al. (2005) Science 308, 1758-1762 (see Example I)), many of which display restricted patterns of expression (see Unigene entries: Hs.91930, Hs.445826, Hs.567737 Mm.276780). Thus, a restricted chemical targeting of microtubules may be possible by inhibition of specific tubulin glutamylases.

Furthermore, ciliates are evolutionary relatives of important apicomplexan parasites (e.g., malaria-causing Plasmodium and Toxoplasma), known to extensively glutamylate pellicular microtubules (Plessmann et al. (2004) Parasitol Res 94, 386-389). Thus narrow specificity inhibitors developed against a Tetrahymena enzyme may be useful as anti-parasite compounds. For example, the eukaryotic parasite Toxoplasma gondii has highly glutamylated pellicular microtubules (Plessmann et al., (2004) Parasitol. Res. 94, 386-389) and Toxoplasma has several glutamylase genes. An inhibitor of a glutamylase can inhibit the growth of, or kill, for example, ciliated protozoans.

An inhibitor of glutamylase can be used in a number of diverse and medically relevant ways. For example, two glutamylases (TTLL6 and TTLL13) in humans are highly expressed in the testis and only at low level or are non-detectable in other tissues (NCBI/Unigene entry Hs.632164; Hs.91930). A mutation in the noncatalytic subunit of one glutamylase, PGs1, blocked assembly of sperm axonemes in the mouse (Campbell et al. (2002). Genetics 162, 307-320). Thus, a glutamylase inhibitor could be used as a male contraceptive, as it can be expected to interfere with the function of sperm flagellum. As shown in the following examples, one of the enzymes in Tetrahymena localizes to cilia, which are structures similar to flagella.

Another potential application is in cancer. It is known that glutamylases modify microtubules of spindles and centrioles in dividing cells (Bobinnec et al. (1998) Cell Motility Cytoskeleton 39, 223-232). Thus, an inhibitor of a glutamylase is expected to inhibit cell proliferation.

Another potential application is as a psychiatric drug. A mutation in the non-catalytic subunit of the glutamylase was found to reduce male-male aggression in the mouse model likely based on its effect on the brain (Campbell et al. (2002). Genetics 162, 307-320). Thus, an inhibitor of glutamylation could be useful as a psychiatric drug.

### EXAMPLES

The present invention is illustrated by the following examples. It is to be understood that the particular examples, materials, amounts, and procedures are to be interpreted broadly in accordance with the scope and spirit of the invention as set forth herein.

### Example I

### Tubulin Glutamylase Enzymes are Members of the TTL Domain Protein Family

Summary

Glutamylation of tubulin has been implicated in several functions of microtubules but the identification of the responsible enzyme(s) has been challenging. We show here that the neuronal glutamylase is a protein complex containing a tubulin tyrosine ligase-like (TTLL) protein, TTLL1. TTLL1 is a member of a large family of proteins with a TTL domain, whose members could catalyze ligations of diverse amino acids to tubulins or other substrates. In the model protist Tetrahymena thermophila two conserved types of glutamylases were characterized, which differ in substrate preference and subcellular localization. Janke et al. (2005) Science 308, 1758-1762.

Introduction

Polyglutamylation is an uncommon type of posttranslational modification that adds multiple glutamic acids to a γ-carboxyl group of a glutamate residue of target proteins, including tubulin and nucleosome assembly proteins NAP1 and NAP2 (Edde et al., Science 247, 83 (1990); Rüdiger et al., FEBS Lett 308, 101 (1992); Redeker et al. FEBS Lett 313, 185 (1992); Regnard et al., J Biol Chem 275, 15969 (2000)). The resulting glutamate side chains are of variable length, allowing for a graded regulation of protein-protein interactions. Glutamylation regulates the binding of neuronal microtubule (MT) associated proteins as a function of the length of the glutamate chain, suggesting that the modification is important for the organization of the neuronal MT network (Bonnet et al., J Biol Chem 276, 12839 (2001)). Tubulin glutamylation may also play a role in centriole maintenance (Bobinnec et al., J Cell Biol 143, 1575 (1998)) axoneme motility (Gagnon et al., J Cell Sci 109, 1545 (1996); Million et al., J Cell Sci 112,4357 (1999)) and mitosis (Bobinnec et al., Cell Motil Cytoskeleton 39, 223 (1998); Regnard et al., J Cell Sci 112, 4281 (1999)).

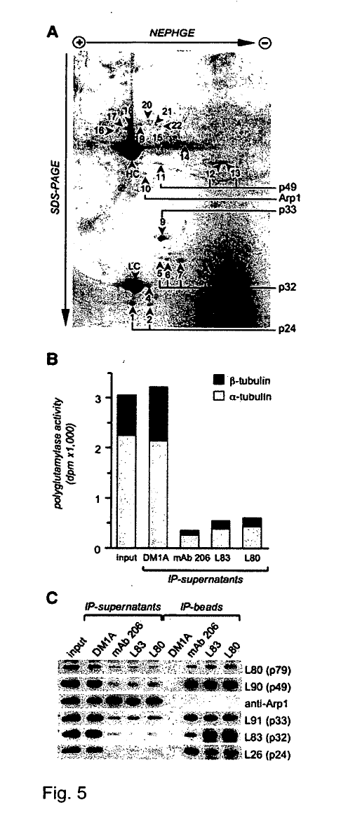

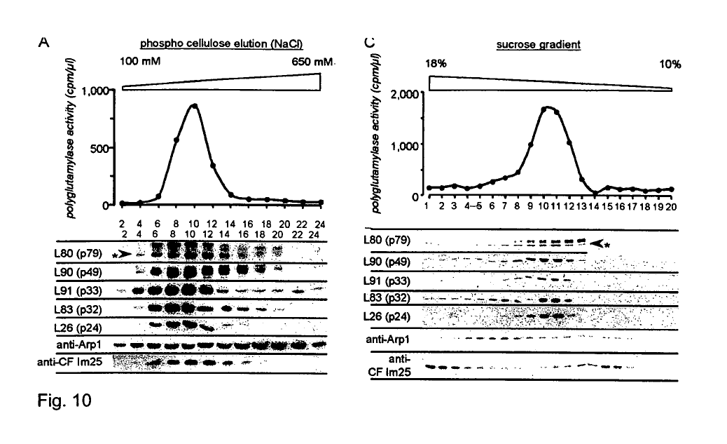

Identification of mouse brain glutamylase. Monoclonal antibody (mAb 206), raised against a partially purified brain tubulin glutamylase fraction, immunoprecipitates the enzyme complex, including the PGs1 protein (Regnard et al., J Cell Sci 116, 4181 (2003)). Here we used gel electrophoresis followed by nano-LC-MS-MS (Liquid Chromatography—Mass Spectrometry) to identify 4 additional protein components of the same complex: p24, p33, p49 and p79 (FIG. 5A; Table I). Polyclonal antibodies were raised against recombinant proteins or peptides of p24, p32/PGs1, p33, p49 and p79 and used for co-immunoprecipitation analyses. mAb 206, anti-p32/PGs1 antibodies (L83) and anti-p79 antibodies (L80) precipitated ≧80% of the glutamylase activity (FIG. 5B) along with all 5 proteins (FIG. 5C). These 5 proteins also consistently copurified with the glutamylase activity during several purification steps (FIG. 10) and were named Glutamylase subunits PGs5, PGs1, PGs2, PGs3 and PGs4 (for p24, p32, p33, p49 and p79, respectively). Additional proteins were found in the mAb 206-immunoprecipitated fraction, including Arp1 and CF Im25 (Table I), but these did not consistently copurify with the enzyme activity.

The apparent size of the neuronal glutamylase complex (360 kDa; Regnard et al., Biochemistry 37, 8395 (1998)) is greater than the theoretical sum of the predicted molecular masses of all 5 subunits (217 kDa), suggesting that the complex contains multiples of one or more subunits. Some of the components appear on 2D gels as multiple spots at different isoelectric points, suggesting that they are themselves subject to charge-altering post-translational modifications (FIG. 5A). Indeed, PGsl was phosphorylated on Ser 279. The 3 more acidic spots of PGs1/p32 contain the phosphorylated peptide 277-284 (RPSVPMAR) (FIG. 5A: spots 5-7). Phosphorylation site prediction (http://www.expasy.org/) indicates that S279 can be phosphorylated by cAMP- or cGMP-dependent protein kinases. The 3 more acidic spots of PGs1/p32 contain the phosphorylated peptide 277-284 (RPSVPMAR) (FIG. 5A: spots 5-7). Phosphorylation site prediction (http://www.expasy.org/) indicates that S279 can be phosphorylated by cAMP- or cGMP-dependent protein kinases.

PGs1 (a product of the mouse gene GTRGEO22) is required for sperm axoneme assembly and normal animal behavior (Campbell et al., Genetics 162, 307 (2002)) and may act in the intracellular targeting of the glutamylase complex (Regnard et al., J Cell Sci 116, 4181 (2003)). PGs3 is an ortholog of the human TTLL1 protein (Trichet et al. Gene 257, 109 (2000)). The amino acid sequence of TTLL1 exhibits 17% identity to tubulin tyrosine ligase (TTL), which catalyses the addition of tyrosine to the C-terminal glutamate of detyrosinated α-tubulin (Ersfeld et al., J Cell Biol 120, 725 (1993)). Despite obvious differences, both glutamylation and tyrosination reactions involve an amino acid addition to a glutamate residue through the formation of an amide bond. Thus, we examined the possibility that TTLL1 is the catalytic subunit of neural tubulin glutamylase.

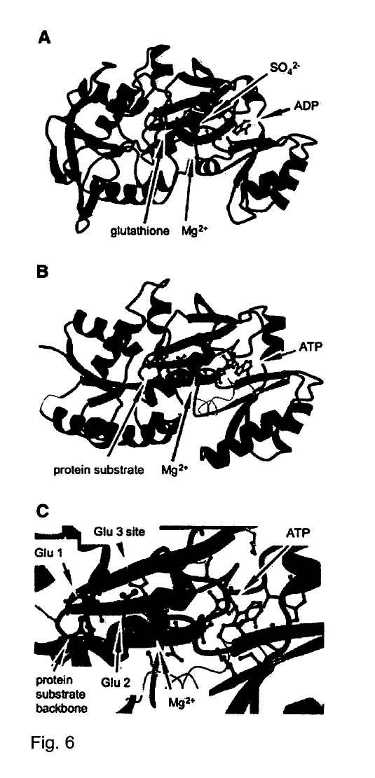

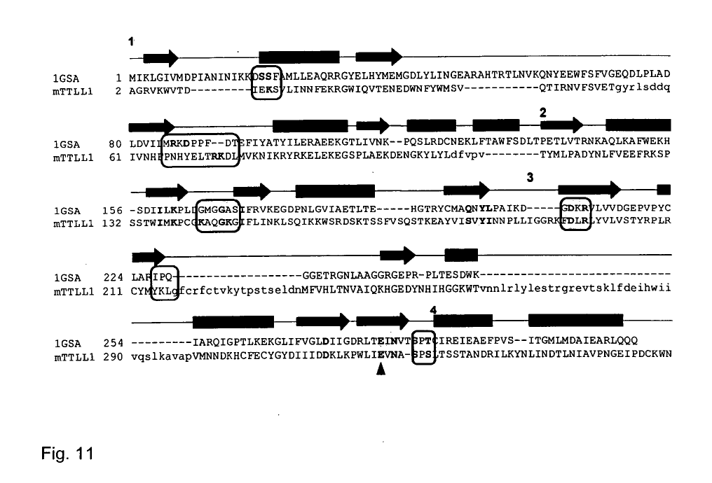

A structural model for TTLL1. The amino acid sequence of TTLL1 (PGs3) contains three conserved motifs which correspond to the ATP/Mg²⁺-binding site typical of enzymes with a carboxylate-amine/thiol ligase activity, such as glutathione synthetase (Galperin et al., Protein Sci 6, 2639 (1997)). Although the overall sequence similarity between TTLL1 and the known carboxylate-amine/thiol ligase enzymes is low, we could align the ATP-binding regions, and also all major parts of TTLL1. A structural model of TTLL1 was obtained by homology-based modeling using glutathione synthetase from E. coli as a template (FIG. 6A, B). Docking of ATP and Mg²⁺ into the model supports the localization of the ATP/Mg²⁺-binding site (FIGS. 6B, C and FIG. 11). We were also able to fit a peptide with a glutamate side chain into the active site, which located a putative binding site for free glutamate (FIG. 6C). Thus, it is very likely that TTLL1 protein is the catalytic subunit of neural tubulin glutamylase.

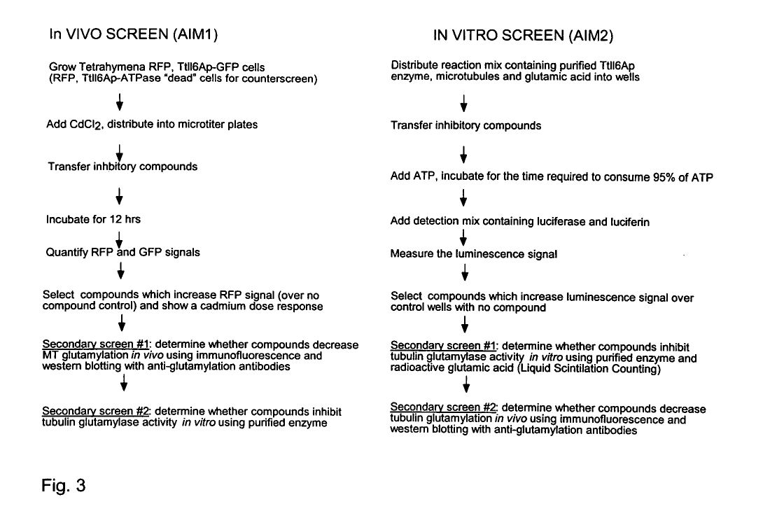

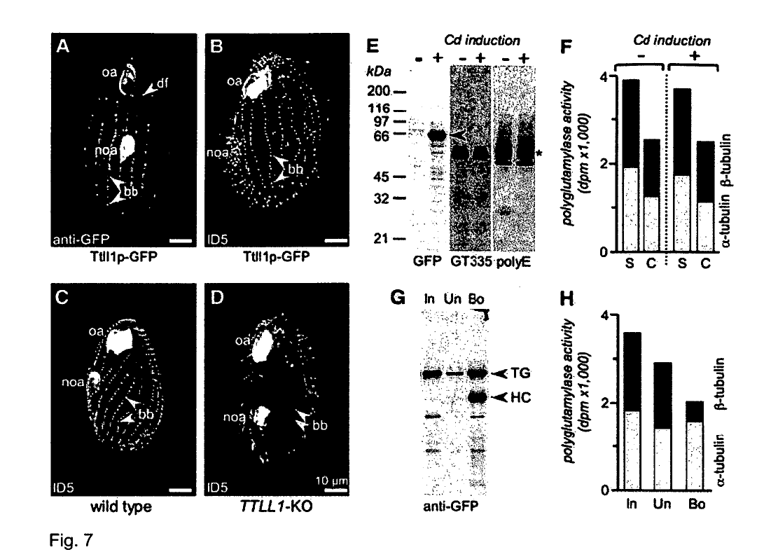

Ttll1p is associated with α-tubulin glutamylase activity in vivo. When expressed in bacteria or in various cell lines as well as in several heterologous systems, the murine TTLL1 had a strong tendency to precipitate and did not show glutamylase activity in vitro. We used a homologous protein expression system based on the ciliated protist Tetrahymena thermophila to assess the role of TTLL1-related proteins in glutamylation. Tetrahymena has a complex cytoskeleton with a large number of distinct types of MTs (Gaertig, J Eukaryot Microbiol 47, 185 (2000)). Most types of MTs in Tetrahymena are monoglutamylated, while a small subset, including MTs of the basal bodies (BBs), cilia, contractile vacuole pore (CVP) and oral deep fiber (DF), have side chains composed of two or more glutamates. Using the recently sequenced macronuclear genome of Tetrahymena, we identified the likely TTLL1 ortholog, Ttll1p (54% of amino acid sequence identity to TTLL1). Ttll1p with an N-terminal GFP was strongly overexpressed using a cadmium-inducible gene promoter (Shang et al., Proc Natl Acad Sci USA 99, 3734 (2002)). Ttll1p-GFP localized to a subset of glutamylated MT organelles: including BBs, CVPs and DF (FIG. 3A), but no increase in the level of MT glutamylation over normal level was detected (FIG. 3B, C, E), and the phenotype appeared normal. Similar results were obtained for the HA epitope C-terminally tagged protein. No change in in vitro tubulin glutamylation activity was detected in cell extracts despite the strong accumulation of Ttll1p-GFP (FIG. 7F). However, a glutamylase activity directed mostly toward α-tubulin was co-immunoprecipitated with anti-GFP antibodies from extracts of overexpressing cells (FIG. 7G, H). The overexpressed protein apparently can replace a part of the endogenous Ttll1p but may not function alone. Based on the data obtained for the murine homolog, it is likely that Ttll1p also acts in a complex and that other subunits are limiting. We also constructed cells completely lacking the TTLL1 gene, using gene disruption. The TTLL1-null cells had a normal phenotype, but showed a strong reduction in tubulin glutamylation in the BBs (FIG. 7C, D), confirming that Ttll1p is involved in glutamylation but also suggesting that there are additional glutamylase activities in this organism that do not require Ttll1p.

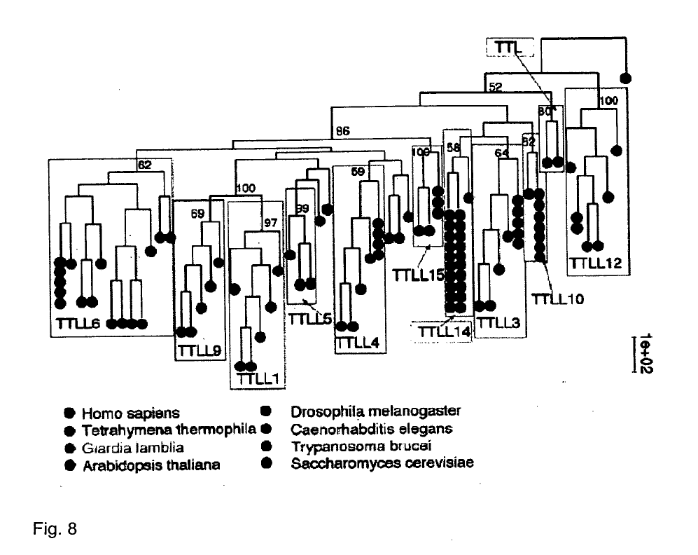

The TTLL family. TTLL1 and TTL are members of a large family of conserved eukaryotic proteins with a TTL homology domain, raising the possibility that other members of this family are also involved in glutamylation or other types of posttranslational amino acid ligations. Phylogenetic analyses showed that TTL-like proteins (TTLLs) of diverse eukaryotes belong to several conserved subtypes (FIGS. 8, 12 and 13). We used the HsTTLL1 sequence (Trichet et al., Gene 257, 109 (2000)) as a template for tBLASTn searches to identify all TTLL loci of Tetrahymena and several other model eukaryotes. Phylogenetic analyses revealed 10 clades of TTLLs, 8 of which contain mammalian proteins. Tetrahymena has between 1-7 sequences in most groups, and one clade of 20 ciliate-specific TTLLs. Among the genomes surveyed, only Trypanosoma has a close homolog of the mammalian TTL, which may be why an enzymatic activity of TTL was not detected in invertebrates (Preston et al., J Mol Evol 13, 233 (1979)) and Tetrahymena (Raybin et al., J Cell Biol 73, 492 (1977)).

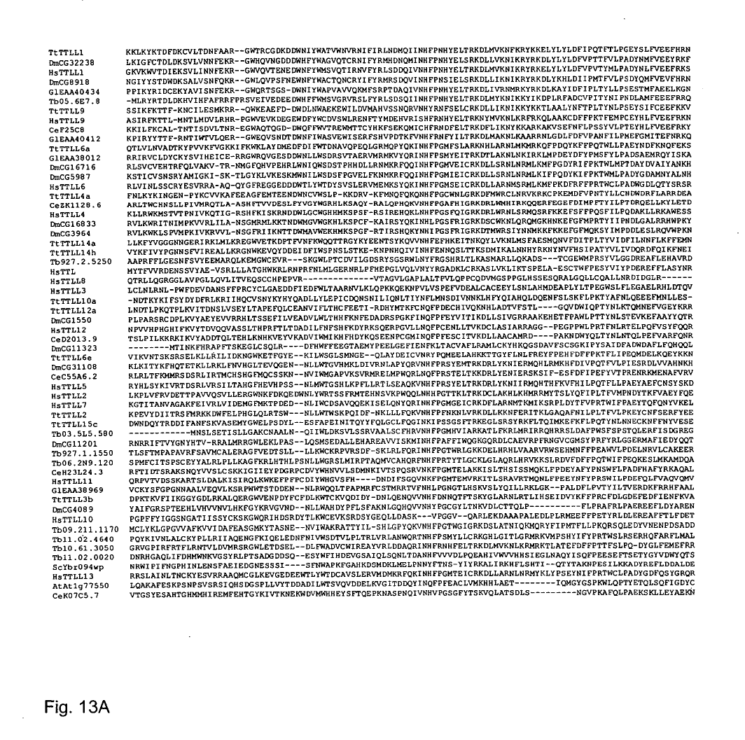

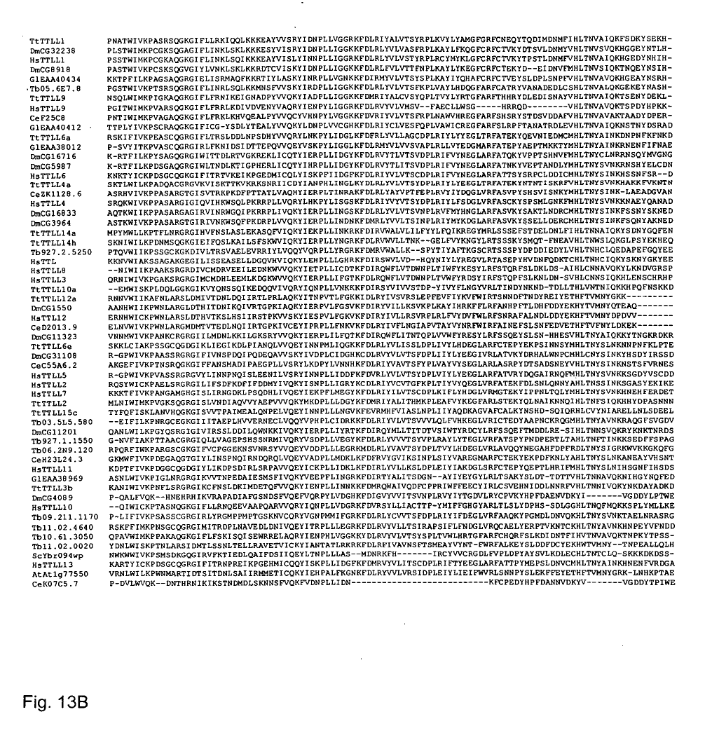

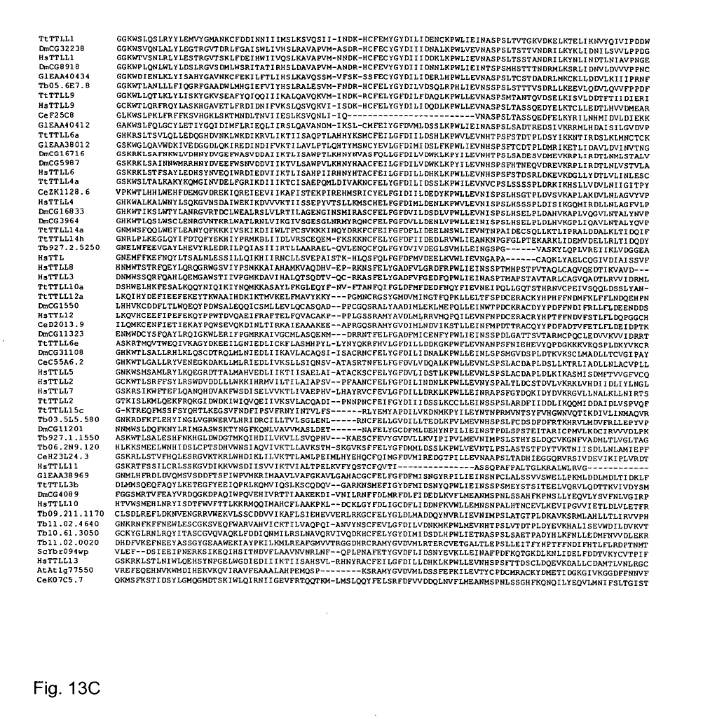

Tetrahymena Ttll6Ap is a β-tubulin-preferring glutamylase. The Tetrahymena TTLL6A sequence belongs to a clade related to TTLL1 type (FIGS. 8 and 13). Overproduced Ttll6Ap-GFP localized mainly to cilia, with a small amount associated with BBs and cell body MTs (FIG. 9A). Overproduction of Ttll6Ap-GFP led to a strong increase in glutamylation in cilia, and on cell body MTs (FIG. 9C; compare FIG. 7C). A strong increase in tubulin glutamylation (but not in polyglycylation) was also detected in whole cells using immunoblotting (FIG. 9F).

A truncated variant lacking the 286 C-terminal amino acids, Ttll6ApΔ₇₁₀-GFP localized predominantly to the cell body with strong preference for subcortical (SC) MTs that extend from the apical end of the cell and run below the BBs (FIG. 9B). Consistently, overexpression of Ttll6ApΔ₇₁₀-GFP led to a strong increase in glutamylation on MTs in the cell body and to a much lesser extent in cilia (FIG. 9D). Thus, the 286 C-terminal amino acids of Ttll6Ap are required for preferential targeting to cilia.

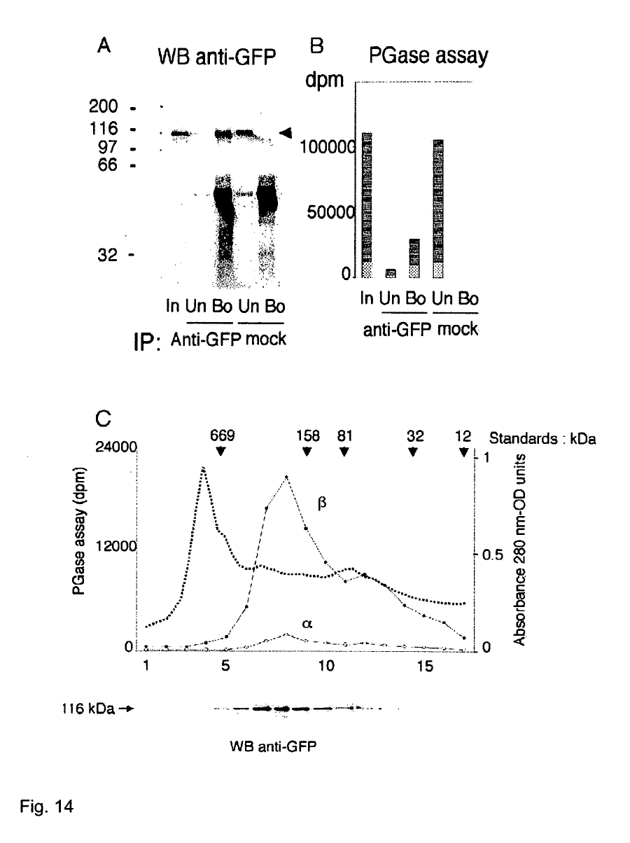

We used the truncated version of Ttll6Ap for biochemical studies, due to the increased presence of this variant in the soluble/cytosolic pool, as compared to the full-length protein. Extracts of cells overexpressing Ttll6ApΔ₇₁₀-GFP showed a 100-fold increase in in vitro glutamylase activity for β-tubulin and a 10-fold increase for α-tubulin compared to non-induced cells (FIG. 9E). This activity co-purified with Ttll6ApΔ₇₁₀-GFP protein under all conditions tested (FIG. 14A-C). No in vitro activity toward NAP proteins was detected. Thus, Ttll6Ap is a tubulin glutamylase displaying a strong preference for the β-tubulin subunit.

Increased glutamylation affects cell growth and motility. Tetrahymena cells overexpressing Ttll6Ap-GFP ceased to multiply in a few hours following cadmium-induction (FIG. 9G) and most had paralyzed cilia (FIG. 9I), indicating that excessive glutamylation inhibits cell proliferation and ciliary dynein-based motility. Cells overexpressing the truncated protein also ceased to proliferate but the effect on ciliary motility was much weaker, in accordance with the altered protein localization pattern (FIG. 9H, I). These effects did not occur when a mutation in the predicted ATP binding site (E422G) of the TTL homology domain was introduced (FIG. 11). Compared to the ATPase-active protein, the inactive variant, Ttll6ApΔ₇₁₀-E422G-GFP, was expressed at a similar level (FIG. 9F) and localized to the same types of MT organelles. However, neither increase in tubulin glutamylation (FIG. 9F), nor alteration of cell growth or motility were observed (FIG. 9H, I), confirming that excessive glutamylation was responsible for the observed effects of Ttll6Ap overproduction.

Conclusion. The simplest interpretation of all our data is that TTLL1/Ttll1p and Ttll6Ap are two types of tubulin glutamylase catalytic components with distinct tubulin subunit preferences. The neuronal TTLL1 as well as Ttll1p have a preference for α-tubulin, while Ttll6Ap preferentially glutamylates β-tubulin. Unlike Ttll6Ap, Ttll1p (and its murine ortholog) did not increase glutamylation in vitro and in vivo upon overproduction. However, TTLL1 exists in a protein complex and additional subunits may be required for its activity and could be limiting in vivo. Indeed, immunoprecipitation of Ttll1p-GFP from overproducing Tetrahymena cells led to a recovery of glutamylase activity.

Ttll6Ap is a much larger protein (116 kDa) compared to TTLL1 (49 kDa) and Ttll1p (42 kDa) and may contain all properties required for autonomous glutamylase activity. The four non-catalytic subunits identified in the neuronal TTLL1 complex may be involved in tubulin substrate recognition, regulation of the enzymatic activity, or subcellular localization as suggested for PGs1 (Regnard et al., J Cell Sci 116, 4181 (2003)). It is likely that Ttll1p is also in a complex, as is the murine homolog. Except for PGs4, we could not identify homologs of the other subunits of the neural complex (PGs1, PGs2, PGs5) outside of vertebrates, including Tetrahymena, indicating that variations in composition of non-catalytic subunits occur across phyla.

The unusually large number of TTLL genes in Tetrahymena and the lack of a detectable loss of function phenotype for TTLL1 suggests functional redundancy. In contrast, a mutation in the PGs1 component of the murine TTLL1-complex lead to defective sperm axonemes and changes in animal behavior (Campbell et al., Genetics 162, 307 (2002)). In C. elegans, RNAi depletion of C55A6.2 (a TTLL5 type) causes embryonic lethality and sterility (Maeda et al., Curr Biol 11, 171 (2001)). Depletion of TTLL1 mRNA in PC 12-E2 cells inhibited neurite outgrowth, suggesting an essential function in neurogenesis. The phylogenetic association of TTLL1, TTLL9, TTLL4, TTLL6, TTLL5 and TTLL15 protein types (86% bootstrap value; FIG. 8) suggests that these protein types are all involved in glutamylation of tubulin or possibly other proteins such as NAPs (Regnard et al., J Biol Chem 275, 15969 (2000)). Other members of the TTLL family may catalyze different types of posttranslational addition of an amino acid, such as polyglycylation.

Materials and Methods

Purification and Identification of Mouse Bain Glutamylase

Glutamylase was partially purified (400×) from 200 3 day-old mouse brains and immunoprecipitated with mAb 206 linked to protein G magnetic beads as described Regnard et al. (J Cell Sci 116, 4181 (2003)). The precipitated proteins were analyzed by two-dimensional (2D) polyacrylamide gel electrophoresis (PAGE). For the first dimension, non-equilibrium pH gradient electrophoresis (NEPHGE; O'Farrell et al. (1977) Cell 12, 1133) and for the second PAGE dimension, an acrylamide gradient (8-13%) were applied. Proteins were stained with Coomassie brilliant blue (Serva) and all visible protein spots (apart from two large spots corresponding to the heavy and light chain of mAb 206) were excised and submitted to in-gel digestion by trypsin. Extracted peptides were analyzed by nano-LC-MS-MS on a Q-TOF 2 mass spectrometer (Micromass Ltd., Manchester, UK) and identified using the Mascot software (Fraering et al. (2004) Biochemistry 43, 9774).

Tetrahymena Cell Extracts

To prepare extracts for glutamylase activity assays, cells were lysed in the T buffer (Tris-HCl 50 mM pH 8.0, NaCl 0.2 M, EGTA 1 mM, MgCl₂ 1 mM, NP-40 0.5%) containing protease inhibitors (leupeptin 0.5 μg/ml, E-64 10 μg/ml, chymostatin 10 μg/ml, antipain 12.5 μg/ml, according to Chilcoat et al. (J Cell Biol 135, 1775 (1996)). Total extracts were either directly analyzed or centrifuged at 50,000×g. The supernatant constituted the soluble fraction. The cytoskeletal fraction was obtained by resuspending and sonicating the pellet in T buffer.

To prepare whole cytoskeletons for western blots, 10⁷ Tetrahymena cells were spun down and processed according to Williams et al. (Methods Cell Biol 47, 301 (1995)) with some modifications. Cells were resuspended in 2 ml of 10 mM Tris-HCl, pH 7.5 with protease inhibitors and incubated on ice for 5 min. An equal volume of 2× lysis buffer with protease inhibitors was added (1% Triton-X100 final concentration) and after 1 min on ice, cytoskeletons were collected by centrifugation (10 min, 16,000 g at 4° C.). Pellets were resuspended in a phosphate buffer and protein concentration was determined using the BCA TM Protein Assay (Pierce). Eight μg of protein was loaded per lane, separated on a 12% SDS-PAGE gel and processed for western blotting.

Size Exclusion Chromatography

A soluble extract of Ttll6ApΔ₇₁₀-GFP producing cells was fractionated on an HPLC TSK G300 SWXL, 7.8×300 mm column (TosoHaas, Pa., USA) in MES 50 mM pH 6.9, EGTA 1 mM, MgCl₂ 1 mM, NaCl 0.35 M, Triton-X100 0.1%, at a flow rate of 0.8 ml/min. Fractions of 0.4 ml were collected. Protein standards used for calibration were thyroglobulin (669 kDa), aldolase (158 kDa), transferrin (81 kDa), hemoglobin (32 kDa) and cytochrome C (12.4 kDa).

Glutamylase Assay

The tubulin glutamylase activity was measured as described Regnard et al. (Biochemistry 37, 8395 (1998)). Briefly, reaction mixtures (20 μl) containing 50 mM Tris-HCl pH 9.0, 2 mM ATP (equilibrated to pH 7.0 with NaOH), 12 mM MgCl₂, 2.5 mM DTT, 10 μM taxotere, L-[³H]-glutamate (45-55 Ci/mmol, Amersham, UK) and 0.1 mg/ml taxotere-stabilized MTs, were incubated at 30° C. for 40 min. In some cases, to increase the radioactive signal, L-[³H]-glutamate was concentrated by centrifugation under vacuum and added at a final concentration of up to 25 μM. Protein fractions to be tested were added at a maximal final concentration of 0.5 mg/ml. The taxotere-stabilized mouse brain MTs were prepared as described Regnard et al. (J Cell Sci 112, 4281 (1999)). The salt concentration was maintained at ≦20 mM to avoid inhibition of the enzyme activity (Regnard et al. (1998) Biochemistry 37, 8395). Quantifications were done by scintillation counting of the α- and β-tubulin bands after SDS-PAGE and electro-transfer onto nitrocellulose, as described Regnard et al. (Biochemistry 37, 8395 (1998)). The specificity of glutamylation to tubulins was verified by submitting the reaction mixtures to SDS-PAGE and fluorography. Exposures were performed at −80° C. using Kodak (Rochester, N.Y.) XAR-5 films after enhancement with Amplify (Amersham). As noted before (Regnard et al. (2003) J Cell Sci 116, 4181), bead fractions obtained after immunoprecipitation have a disproportionally low activity compared to the input or unbound fractions, presumably because the beads hinder proper interactions between the glutamylase and the MT substrate. Therefore, 10-20 times more of the bead material was used compared to the equivalent amount of unbound and input material, but the levels of activity remained relatively low. Thus, the glutamylase assays of bead fractions cannot be considered as quantitative; they only provide qualitative information about the type of activity present. However, relevant quantitative information can be deduced from the comparison of the activities in the input and unbound material.

Antibodies

Mouse monoclonal antibodies (mAbs) used were mAb 206 (Regnard et al. (2003) J Cell Sci 116, 4181), GT335 (anti-glutamylation; Wolff et al., Eur J Cell Biol 59, 425 (1992)), ID5 (anti-glutamylation; Rudiger et al., Eur J Cell Biol 78, 15 (1999)), 12G10 (anti-α-tubulin, from J. Frankel).

Polyclonal rabbit Abs were anti-Arp1 (courtesy of R. Melki), anti-CF Im25 (courtesy of S. Dettwiler and W. Keller), anti-GFP (Torrey Pines Biolabs, Houston, Tex.), polyE anti-glutamylation (serum 2303) and polyG anti-polyglycylation (serum 2301), the latter two courtesies of M. Gorovsky.

Laboratory-made rabbit polyclonal Abs were L26 (anti-p24/PGs5), L83 (anti-p32/PGs1; Regnard et al. (2003) J Cell Sci 116, 4181), L91 (anti-p33/PGs2 anti-peptide Ab), L90 (anti-p49/PGs3) and L80 (anti-p79/PGs4 anti-peptide Ab).

Secondary antibodies used were: HRP-labeled donkey anti-rabbit Ig 1:10,000, HRP-labeled protein A 1:5,000 (Amersham Pharmacia Biotech), goat-anti-mouse FITC and goat-anti-rabbit FITC (Zymed).

Immunoprecipitation

For immunoprecipitation, antibodies (FIGS. 5, 7, 14) were pre-incubated with protein G magnetic beads (Dynal Biotech), washed in PBS with 0.05% Tween-20 and incubated with the input material for 3 hrs at 4° C. The beads were extensively washed with Tris-HCl 50 mM pH 8.0, NaCl 0.5 M, NP-40 0.1% before analysis.

Western Blotting

For western blots shown in FIG. 5, antibodies were used at 0.5 μg/ml. For FIGS. 7 and 14, anti-GFP, GT335 and anti-polyE antibodies were diluted at 1:5,000, 1:20,000 and 1:3,000, respectively. For western blots presented in FIG. 9 antibodies were used at the following dilutions: ID5 (1:100), polyG 2301 (1:2,000), 12G10 (1:100), anti-GFP (1:1,000). Proteins were visualized with HRP-labeled donkey anti-rabbit Ig 1:10,000 or HRP-labeled protein A 1:5,000, followed by detection with chemo luminescence (Western Lightning Chemo-luminescence Reagent Plus, Perkin Elmer).

Modeling of the Structure of TTLL1

TTLL1 (PGs3) contains an ATP/Mg²⁺-binding site typical of enzymes with an ATP-dependent carboxylate-amine/thiol ligase activity (Galperin et al. (1997) Protein Sci 6, 2639; Dideberg et al. (1998) Trends Biochem Sci 23, 57). These so-called ADP-forming enzymes catalyze ATP-dependent ligations of carboxyl group carbon atoms of the first substrate to an amino (or imino) acid group nitrogen atoms of the second substrate via the formation of acylphosphate intermediates, as glutamylation reaction is predicted to involve. Previous structural alignments of ADP-forming enzymes have identified three conserved sequence motifs that correspond to the ATP-binding cleft (Artymiuk et al. (1996) Nat Struct Biol 3, 128), which are also well conserved in TTLL1. In enzymes with a known structure, these conserved regions are located at the ATP/Mg²⁺-binding sites. Nevertheless, about 85% of the sequence of the ATP/Mg²⁺-binding regions of the structurally characterized enzymes differs from TTLL1. To test our hypothesis about the enzymatic function of TTLL1, we applied a combination of a structure-based alignment, sequence profile search and molecular modeling. Several structures of ADP-forming enzymes (pdb codes: 1GSA, 1BNC, 2DLN, 1MOW, 1EHI, 1JDB, 1EYZ) were superposed with the InsightII program (Dayring et al. (1986) J Mol Graph 4, 82). The initial CLUSTAL alignment of the enzyme sequences was manually improved according to structural superposition. The resulting sequence alignments served for the generation of sequences profiles with the generalized sequence profile method and the pftools package (Bucher et al. (1996) Comput Chem 20, 3). The probability of random alignment was calculated by analyzing the score distribution obtained from a profile search versus a regionally randomized version of the protein database, assuming an extreme value distribution (Hofiann et al. (1995) Trends Biochem Sci 20, 347). Apart from the central ATP-binding site, the structures of all analyzed enzymes did not match. This changed when the enzyme structures were divided into four sub-structures (N-terminal substrate-binding domain, part 1 of ATP-binding domain, part 2 of ATP-binding domain and C-terminal region; FIG. 11) and separately superimposed with each other, yielding four structure-based alignments with corresponding sequence profiles. These sequence profiles (Bucher et al. (1996) Comput Chem 20, 3) were then applied to the sequence of TTLL1.

The initial structure of TTLL1 was constructed using the HOMOLOGY module of InsightII program (Dayring et al. (1986) J Mol Graph 4, 82) based on the alignment of TTLL1 to the known 3D-structures of ADP-forming enzymes, but mainly to glutathione synthetase (pdb code: 1GSA; Hara et al., Biochemistry 35, 11967 (1996)). The resulting model was subjected to 300 steps of minimization based on the steepest descent algorithm with the backbone atoms of α-helical segments restrained to their starting positions with the force constant K=100. The next 500 steps of the refinement were performed without any restrictions, using conjugate gradients algorithm. The CHARMM force field (Brooks et al. (1983) J Comp Chem 4, 187) and the distance dependent dielectric constant were used for the energy calculations. The program PROCHECK (Laskowski et al. (1993) J Appl Crystallog 26, 282) was used to verify the quality of the modeled structure. The figures were generated with the programs Molscript and Raster3D (FIG. 6) (Kraulis et al. (1991) J Appl Crystallog 24, 946; Bacon et al. (1988) J Mol Graph 6, 219). As a result, we obtained an alignment of TTLL1 not only in the previously mentioned ATP-binding regions, but also in all major parts of the protein (FIG. 11) with a structure highly similar to glutathione synthetase. ATP, Mg²⁺ and a peptide with a glutamate side chain were docked into the model taking into consideration an analogous molecular co-crystallization with know ATP-dependent carboxylate-amine ligases (FIG. 6). For simplification of the model, we have fitted an A-E-A peptide modified on the central E into the structure, however, we have before shown that the flanking A residues could be replaced by others without changes for the fitting.

Searching of Genonies and Phylogenetic Analyses

The Arabidopsis thaliana, yeast, human and Giardia lamblia predicted TTL domain protein sequences were identified by BLAST searches of the NCBI databases (http://www.ncbi.nlm.nih.gov/BLAST/). The Drosophila melanogaster sequences were identified by BLAST searches at the FlyBase (http://flybase.bio.indiana.edu/). The Caenorhabditis elegans, and Trypanosoma brucei sequences were identified using The Sangers Institute (http://www.sanger.ac.uk/DataSearch/) and The Institute for Genomic Research (TIGR) databases for respective species (http://www.tigr.org). The Tetrahymena thermophila TTL domain sequences were identified by tBLASTn searches of the recently assembled macronuclear genome at the TIGR Tetrahymena blast server (http://tigrblast.tigr.org/er-blast/index.cgi?project=ttg) using genomic scaffolds of the November 2003 assembly. Using HsTTLL1 we first identified the Tetrahymena TTLL1 homolog gene and exhaustive searches of the Tetrahymena genome were continued using TTLL1 and several other TTL domain sequences. Starting from the region of homology to TTL domains of other organisms, the probable coding region was reconstructed based on the higher G/C content. The intron junctions were tentatively identified based on the known consensus splice sites (Wuitschick et al. (1999) J Eukaryot Microbiol 46, 239). As we have completed the manual prediction of the coding regions, the Tetrahymena Genome Project at TIGR has published the preliminary gene predictions based on gene finder programs. In general there was an agreement between the manual and software-based predictions. We verified each putative TTLL sequence by a reverse BLASTp of the NCBI databases. A cDNA sequence of TTLL4A was kindly provided by Drs. Kathleen Clark and Martin Gorovsky (University of Rochester, N.Y.). The cDNA plasmid of TTLL14F was kindly provided by Dr. Aaron Turkewitz (University of Chicago, IL).

The ends of the transcribed regions of TTLL1 and TTLL6A genes were identified by PCR of cDNAs pools or cDNA libraries from Tetrahymena cells grown under several different conditions. Total RNA was isolated using the modified TRI Reagent procedure (MRC; Chomczynski et al. (1995) Biotechniques 19, 942). cDNA was synthesized using primers of the SMART cDNA library construction kit and the PowerScript Reverse Transcriptase (Clontech). One μg of total RNA was used for the first strand cDNA synthesis (1.5-2 hrs at 42° C.). Next, 1 μl of the first strand cDNA reaction mix was used for a PCR reaction with the same primers (extension step at 68° C. for 8 min). A cDNA from cilia-regenerating cells was used to construct a small cDNA library (43,000 clones) by cloning into a plasmid vector according to the Clontech kit instructions. The 5′ end of TTLL1 UTR was determined by 5′RACE PCR of the cDNA library using a gene specific primer anchored inside the conserved region and the SMART IV primer (Clontech). The 3′ end of the TTLL1 cDNA was identified by 3′ RACE PCR using a gene-specific primer and the CDS III/3′ primer (Clontech). The same 5′ and 3′ RACE strategy with the cDNA library as a template was used to obtain terminal fragments of cDNAs for TTLL6. The fragment of cDNA initially amplified for the 3′ end of the TTLL6 UTR lacked a stop codon in the translational frame, suggesting that the cDNA template was truncated. The protein prediction TTLL6A produced by the TIGR gene finder software indicated that the coding region extends for another 286 codons. Using a primer walking strategy with a pool of cDNA we subsequently mapped the end of TTLL6A transcribed region at a more downstream position, consistent with the TIGR prediction.

The TTL domains of each predicted protein sequence were identified manually using highly conserved peptide motifs and a preliminary alignment was done using Clustal X 1.82 program (Jeanmougin et al. (1998) Trends Biochem Sci 23, 403) followed by extensive manual adjustment using the Seaview program (Galtier et al., (1996) Comput Appl Biosci 12, 543). Phylogenetic analyses were performed using the Phylip version 3.6 package (Felsenstein, (1997) Syst Biol 46, 101) using the following programs. One hundred replicates of the sequence set were created using SEQBOOT. The distances were calculated using PROTDIST, trees were reconstructed using NEIGHBOR. The Jones-Taylor-Thorton (JTT) substitution model was used. A consensus tree was obtained using CONSENSE and the tree was plotted using DRAWGRAM.

Gene Knockout in Tetrahymena

A 1.9 kb 5′ untranslated region immediately upstream of the TTLL1 coding region was amplified using the KOS1 (5′-ATTTTATGAGCTCCACCATCTTTTATTTTGCTTT-3′) and KOA1 (5′-TAAATAAGGATCCACACAAAATAGATAAAAAGGAG-3′) primers. SacI and BamHI restriction sites were introduced at the ends of the above primer sequences, respectively. The PCR product was digested with BamHI and SacI and cloned into the p4T2-1 plasmid on one side of the neo2 cassette. A 1.6 kb fragment of the 3′ UTR of TTLL1 containing a part of TTLL1 coding region was amplified using the TTL-KO2-F (5′-ATTTTATATCGATTTGTT AAACCAGCATCACGA-3′) and TTL-KO2-R (5′-TAAATAACTCGAGAAAATTAAATGTCTGGCTGGAT-3+) primers. The ClaI and XhoI restriction sites were introduced at the ends of the primers and used for subcloning into the other side of the neo2 cassette of p4T21. The resulting gene knockout targeting plasmid named pTTLL1-KO was digested with SacI and AhoI to release the disruption fragment of TTLL1::neo2 from the plasmid backbone. The germline disruption of the TTLL1 gene was done by biolistic bombardment and the knockout heterokaryons were constructed as described in Cassidy-Hanley et al. (Genetics 146, 135 (1997)). Cells homozygous for the TTLL1 deletion were obtained by a cross of two TTLL1 knockout heterokaryons (Hai et al. (2000) Methods Cell Biol 62, 513).

Expression and Localization of GFP Fusion Proteins in Tetrahymena

The pMTT1-GFP-N vector suitable for expression of coding regions with an N-terminal GFP tag under control of the MTT1 promoter was constructed on the basis of the pMTT1-IFT52-GFP plasmid (Brown et al. (2003) Mol Biol Cell 14,3192). The coding region of IFT52 of this plasmid was replaced by the coding region of GFP (Haddad et al. (1997) Proc Natl Acad Sci USA 94, 10675) using a forward primer with a HindIII site and a reverse primer with MluI-BamHI sites (and a TGA stop codon between these two sites). Subcloning a coding region between the HindIII and BamHI sites of the resulting pMTT1-GFP-N provides a fragment which can be integrated into the BTU1 locus of Tetrahymena and the coding region can be expressed using the MTT1 promoter fragment in response to cadmium (Shang et al. (2002) Proc Natl Acad Sci USA 99, 3734). To overexpress TTLL1, the entire predicted coding region of TTLL1 gene was amplified from a total genomic DNA with addition of an MluI site to the forward primer and a BamHI site to the reverse primer and subcloned into pMTT1-GFP-N to create pMTT1-GFP-TTLL1. The same strategy was used to amplify the predicted coding region of TTLL6A to create pMTT1-GFP-TTLL6L plasmid, except that the reverse primer was designed to anchor at the position of 390 bp downstream of the predicted TGA stop codon. Thus, after integration into the Tetrahymena genome, using the pMTT1 -GFP-TTLL6L fragment, the TTLL6A coding region is probably expressed using the native 3′ nontranscribed region. To create a plasmid for expression of the truncated version of Ttll6Ap gene product (Ttll6ApΔ₇₁₀-GFP), the coding region encompassing the amino acid codons 1-710 was amplified with MluI and BamHI sites added at the ends of the primers and subcloned into pMTT1-GFP-N to create pMTT1-GFP-TTLL6S. To produce a plasmid for expression of the ATPase deficient form of Ttll6ApΔ₇₁₀-GFP, we changed the predicted codon 422 from glutamic acid to glycine using the QuickChange site-directed mutagenesis kit (Stratagene) and obtained the plasmid named pTTLL6Δ-GFP.

For expression of GFP fusion proteins in Tetrahymena, we used the negative selection method based on targeting to the BTU1 locus (Gaertig et al. (1999) Nat Biotechnol 17, 462). All plasmids used for expression of GFP fusions had fragments of the BTU1 locus flanking sequences for homologous targeting. Following biolistic bombardment of starved CU522 cells, transformants with integrations into the BTU1 locus were selected with 20 μM paclitaxel and the presence of a transgene was confirmed by PCR using total genomic DNA. The expression of the transgene was induced by adding 2.5 μg/ml cadmium chloride to the medium and cells were analyzed microscopically or biochemically 2-4 hrs later.

Immunofluorescence and Microscopy

For immunofluorescence, cells were processed as described in Thazhath et al. (Nat Cell Biol 4, 256 (2002)) with modifications. Briefly, we hand-picked 50-100 cells using a pipette, washed cells by releasing into a drop of 10 mM Tris-HCl, pH 7.5 buffer on a cover slip. Cells were permeabilized on cover slips for 45-60 sec by exposure to 10 μl of 0.5% Triton-X100 in the PHEM buffer (Gaertig et al. (1992) Protoplasma 167, 74), followed by addition of 15 μl of 2% paraformaldehyde in the PHEM buffer. The fixed cells were air-dried and coverslips incubated for 10 min in the blocking solution (3% bovine serum albumin (BSA) in phosphate-buffered saline (PBS) with 0.1% Tween-20) and incubated for a few hours at room temperature (or overnight at 4° C.) in primary antibodies in PBS with 3% BSA and 10% normal goat serum. The following primary antibodies were used at the indicated dilutions: anti-GFP (1:100), and ID5 (1:10). The cover slips were washed 3 times for 5 min by immersion in PBS using small staining jars followed by incubation in the secondary antibodies diluted 1:100 for 1 hr at room temperature. After 3 washes cover slips were mounted as described in Gaertig et al. (Protoplasma 167, 74 (1992)). To visualize only Ttll6Ap-GFP fluorescence, cells were fixed as described above and analyzed directly. Images were collected using a Leica TCS SP2 Spectral Confocal Microscope with Coherent Ti: sapphire multiphoton laser (Mira Optima 900-F). Usually a series of optical sections was obtained and the top half of the sections was assembled into a composite image.

FIG. 10 describes co-purification of the glutamylase subunits and enzymatic activity. Fractions of the two main purification steps of glutamylase from brain extract, phospho cellulose chromatography (A, B) and sucrose gradient centrifugation (C, D), are analyzed for glutamylase activity (A, C) and by western blot for the proteins p24, p32, p33, p49, p79, Arpl and CF Im25 (B, D). The five subunits p24 to p79 all eluted together with the peaks of enzymatic activity (A, C). CF Im25 and Arp1 show partial co-elution with glutamylase in the phospho cellulose step, but were mostly recovered in other fractions of the sucrose gradient. Note that L80 cross-reacts on western blots with another protein band of slightly higher apparent mass than p79, which is not immunoprecipitated (* the arrow points to the specific protein band for p79).

FIG. 11 shows a sequence profile alignment for TTLL1 modeling. The sequences of glutathione synthetase from E. coli (1GSA) and TTLL1 from mouse (mTTLL1) have been aligned. The elements of the secondary structure, α-helices (turquoise arrows) and β-sheets (violet boxes), are shown above the alignment. Amino acids in lower case (mTTLL1) stand for regions that were not modeled. Red numbers indicate the beginning of four sub-domains: (1) N-terminal substrate-binding domain, (2) part 1 and (3) part 2 of the ATP-binding domain and (4) the C-terminal region. Residues that interact with ATP and Mg²⁺ are in bold letters and the corresponding domains are outlined in yellow. All residues of the active site that are conserved with other ATP-dependent carboxylate-amine ligases are written in dark green (see FIG. 6C). Residues located in the proximity of the substrates are outlined in green. The E326 residue (mTTLL1) indicated by an arrowhead corresponds to the matched E422 residue of Ttll6Ap that has been mutated in Ttll6ApΔ₇₁₀-E422G.

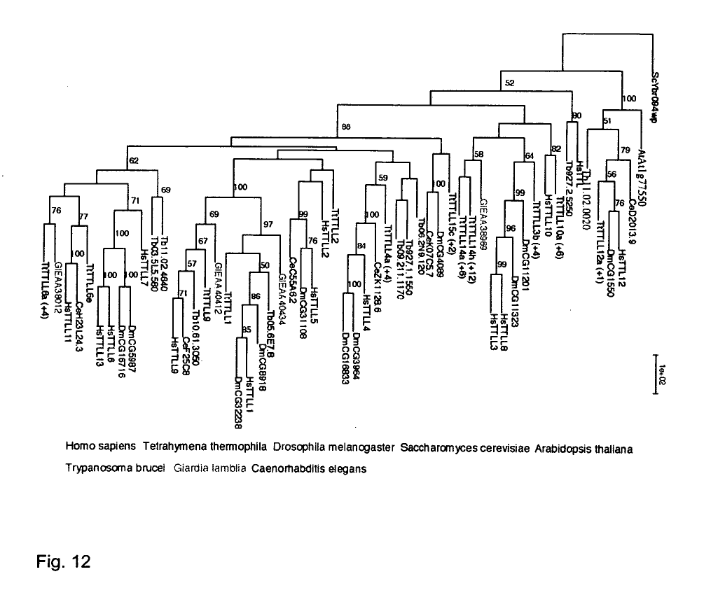

FIG. 12 presents a phylogenetic analysis of predicted protein gene sequences containing a TTL domain. The TTL domains of each predicted protein sequence were aligned was using Clustal X 1.82 followed by manual adjustment in SeaView. Phylogenetic analyses were performed using the Phylip version 3.6 package. One hundred replicates of the sequence set were created using SEQBOOT. The distances were calculated using PROTDIST, and trees were constructed using NEIGHBOR. The Jones-Taylor-Thorton (JTT) substitution model was used. A single consensus tree was obtained using CONSENSE. The yeast sequence was used as an outgroup. Tetrahymena has an extraordinarily large number of predicted TTL domain genes (50). Only one paralog of Tetrahymena is shown in each orthologous group and the number of additional paralogs is indicated in parentheses. The following are GenBank accession numbers of predicted protein sequences used in the analysis. Some sequences are not yet deposited in GenBank including some of the Trypanosoma brucei genes (whose sequences were derived from TIGR annotations) and most Tetrahymena sequences (also derived from the genomic sequences identified using the TIGR Tetrahymena blast server. Homo sapiens: TTL (Q8NG68), TTLL1 (NP_{—}036395), TTLL2 (AAH30650), TTLL3 (T12515), TTLL4 (NP_{—}055455), TTLL5 (NP_{—}05587), TTLL6 (BAC05032), TTLL7 (AAH60878), TTLL8 (XP_{—}104657), TTLL9 (XP_{—}092778), TTLL10 (BAC85781), TTLL11 (AAM81328), TTLL12 (AAH01070), TTLL13 (XP_{—}496092). Drosophila melanogaster: CG32238 (NP_{—}729025), CG31108 (NP_{—}733081), CG16833 (NP_{—}723643), CG16716 (NP_{—}725916), CG11201 (NP_{—}609068), CG11323 (NP_{—}609069), CG8918 (NP_{—}573197), CG5987 (NP_{—}651549), CG4089 (NP_{—}650021), CG3964 (NP_{—}722946), CG1550 (NP_{—}610325). Saccharomyces cerevisiae: Ybr094wp (NP_{—}009652.1). Arabidopsis thaliana: At1g77550 (NP_{—}177879.2). Trypanosoma brucei: Tb927.1.1550 (CAB95431), Tb927.2.5250 (XM_{—}340623) Tb05.6E7.820 (TIGR annotation 314.m00409), Tb06.2N9.120 (TIGR 315.m00360) Tb03.5L5.580 (TIGR 312.m00548), Tb09.211.1170 (TIGR 320.m00798) Tb11.02.4640 (322.m00560), Tb10.61.3050 (TIGR 319.m01288) Tb11.02.0020 (322.m00126). Caenorhabditis elegans: F25C8 (part of the F25C8 cosmid, Sanger Institute, UK), C55A6.2 (NP_{—}505918), ZK1 128.6 (CAA87425), H23L24.3 (AAL06035), K07C5.7 (CAA94900), D2013.9 (CAA87783). Giardia lamblia: GLP_{—}43_{—}54366_{—}55577 (EAA40434), GLP_{—}43_{—}15991_{—}17301 (EAA40412.1), GLP_{—}618_{—}12970_{—}14472 (EAA38012.1), GLP_{—}205_{—}13412_{—}15397 (EAA38969.1), GLP_{—}223_{—}2566_{—}4779 (EAA37058.1), GLP_{—}251_{—}3885_{—}7112 (EAA38326.1), GLP_{—}203_{—}36475_{—}34136 (EAA39544.1).

The following are scaffold designations for the Tetrahymena TTLL gene sequences using the November 2003 assembly of the Tetrahymena macronuclear genome by TIGR: TTLL1 (8254582), TTLL2 (8254617), TTLL3A (8254579), TTLL3B (8254645), TTLL3C (8254670), TTLL3D (8254548), TTLL3E (8254652), TTLL4A (8254630), TTLL4B (8254717), TTLL4C (8254496), TTLL4D (8254359), TTLL4E (8254495), TTLL6A (8254600), TTLL6B (8254747), TTL6C (8254577), TTLL6D (8254607), TTLL6E (8254650), TTLL6F(8254010), TTLL9 (8254449), TTLL10A (8254580), TTLL10B (8254751), TTLL10C (8254034), TTLL10D (8254666), TTLL10E (8254504), TTLL10F (8254563), TTLL12A (8254576), TTLL12B (8254820), TTLL14A (8254487), TTLL14B (8254479), TTLL14C (8254589), TTLL14D (8254379), TTL14E (8254555), TTLL14F (8254814), TTLL14G (8253864), TTLL14H (8254469), TTLL14I (8254688), TTLL14J (8254798), TTLL14K (8254403), TTLL14L (8254823), TTLL14M (8254557), TTLL14N (8254748), TTLL14O (8254609), TTLL14P (8254819), TTL14Q (8254545), TTLL14R (8254737), TTLL14S (8254811), TTLL14T (8254373), TTLL15A (8254385), TTLL15B (8254460), TTLL15C (8254459).

FIG. 13 shows a multiple sequence alignment of TTL domains of TTL and TTLL proteins. This alignment was used to construct the tree shown in FIGS. 8 and 12. Regions with low homology were not included in the alignment. Red: amino acid identity, blue: amino acid similarity (Clustal X alignment was colored with BoxShade http://www.ch.embnet.org/software/BOX_form.html). At: Arabidopsis thaliana, Ce: Caenorhabditis elegans, Dm: Drosophila melanogaster, Gl: Giardia lamblia, Hs: Homo sapiens, Sc: Saccharomyces cerevisiae, Tb: Trypanosoma brucei, Tt: Tetrahymena thermophila.

FIG. 14 shows glutamylation activity of Ttll6Ap in vitro. Glutamylase activity co-purified with Ttll6ApΔ₇₁₀-GFP protein under all conditions tested, including immunoprecipitation (A, B), phosphocellulose (results not shown) and size exclusion chromatography (C). A-B. Soluble extracts of Ttll6ApΔ₇₁₀-GFP. expressing cells were submitted to immunoprecipitation with anti-GFP antibodies or unrelated rabbit antibodies (mock). The input (In) as well as the unbound (Un) and bound (Bo) fractions were analyzed by western blot with anti-GFP antibody (A) and assayed for tubulin glutamylase activity (B). Equal proportions of input, unbound and bead fractions were analyzed by western blotting, while 10 times more bead fraction was assayed for glutamylase activity. C. Size exclusion chromatography using a soluble fraction of cells expressing Ttll6ApΔ₇₁₀-GFP. A soluble extract (4 mg of protein) of induced Ttll6ApΔ₇₁₀-GFP expressing cells was fractionated on an HPLC TSK column. Each fraction was analyzed by western blotting with anti-GFP antibodies (lower panel) and assayed for tubulin glutamylase activity using adult brain MTs as substrate (radioactive glutamate concentration 5 μM). The results are expressed as dpm incorporated into α-(open circles) and β-tubulin (filled circle). The absorbance profile at 280 nm is shown (dashed line). Arrowheads indicated the elution peak position of standard proteins used for calibration. The enzymatic activity and Ttll6ApΔ₇₁₀-GFP co-eluted in a peak of 180 kDa, while the expected size of Ttll6ApΔ₇₁₀-GFP is 116 kDa. It is possible that the Ttll6ApΔ₇₁₀-GFP is a homodimer, especially because the N-terminal region of the predicted peptide sequence contains a coiled-coil region.

TABLE I
Identification of all proteins precipitated with mAb 206 (see FIG. 5A).

| spot | | Genebank | recovered | MASCOT | |
|---|---|---|---|---|---|
| N^{oA)} | identified protein^{B)} | accession N^{oB)} | sequence^{B)} | score^{B)} | protein identity |
| p79^{C)} | D430025H09Rik protein | 16741525 | 26% | 622 | p79/PGs4 |
| 1 | nicolin 1^{D)} | 13384852 | 42% | 315 | p24/PGs5 |
| 2 | nicolin 1^{D)} | 13384852 | 48% | 404 | p24/PGs5 |
| 3 | cleavage and polyadenylation specific factor 5, | 13386106 | 23% | 190 | CF Im25 |
| | 25 kD subunit | | | | (Ruegsegger et al., |
| | | | | | Mol Cell 1, 243 |
| | | | | | (1998)) |
| 3 | complement component 1, q subcomponent, | 6671652 | 33% | 236 | non-specific |
| | gamma polypeptide | | | | |
| 4 | chain A, IgG2a Fab fragment | 640171 | 15% | 198 | non-specific |
| 5 | gene trap ROSA b-geo 22 | 22507341 | 32% | 496 | p32/PGs1 |
| 6 | gene trap ROSA b-geo 22 | 22507341 | 34% | 496 | p32/PGs1 |
| 7 | gene trap ROSA b-geo 22 | 22507341 | 57% | 607 | p32/PGs1 |
| 8 | gene trap ROSA b-geo 22 | 22507341 | 38% | 572 | p32/PGs1 |
| 9 | RIKEN cDNA 5730494M16 | 25029762 | 43% | 384 | p33/PGs2 |
| 10 | ARP1 actin-related protein 1 homolog A, | 5031569 | 20% | 287 | Arp1 (Holleran et al., |
| | centractin alpha | | | | J Cell Biol 135, 1815 |
| | | | | | (1996); Schafer et al., |
| | | | | | J Cell Biol 126, 403 |
| | | | | | (1994)) |
| 11 | tubulin tyrosine ligase-like 1 | 30725861 | 18% | 321 | p49/PGs3 |
| 12 | tubulin tyrosine ligase-like 1 | 30725861 | 30% | 428 | p49/PGs3 |
| 13 | tubulin tyrosine ligase-like 1 | 30725862 | 45% | 531 | p49/PGs3 |
| 14 | similar to Ig gamma-2B chain C region secreted | 28523035 | 35% | 156 | non-specific |
| | form | | | | |
| 14 | fibrinogen, B beta polypeptide | 20872398 | 44% | 665 | non-specific |
| 15 | immunoglobulin heavy chain constant region | 14091948 | 21% | 198 | non-specific |
| 15 | fibrinogen, B beta polypeptide | 20872398 | 30% | 552 | non-specific |
| 16 | chaperonin subunit 8 (theta) | 6753328 | 43% | 1110 | TRIC component |
| | | | | | (Lopez-Fanarraga et |
| | | | | | al., J Struct Biol 135, |
| | | | | | 219 (2001); Leroux et |
| | | | | | al., Curr Biol 10, |
| | | | | | R260 (2000)) |
| 17 | chaperonin containing TCP-1 theta subunit | 5295992 | 23% | 531 | TRIC component |
| | | | | | (Lopez-Fanarraga et |
| | | | | | al., J Struct Biol 135, |
| | | | | | 219 (2001); Leroux et |
| | | | | | al., Curr Biol 10, |
| | | | | | R260 (2000)) |
| 18 | chaperonin subunit 5 (epsilon) | 6671702 | 10% | 225 | TRIC component |
| | | | | | (Lopez-Fanarraga et |
| | | | | | al., J Struct Biol 135, |
| | | | | | 219 (2001); Leroux et |
| | | | | | al., Curr Biol 10, |
| | | | | | R260 (2000)) |
| 19 | keratin 1 | 17318569 | 25% | 620 | contamination |
| 20 | matricin | 631730 | 30% | 640 | TRIC component |
| | | | | | (Lopez-Fanarraga et |
| | | | | | al., J Struct Biol 135, |
| | | | | | 219 (2001); Leroux et |
| | | | | | al., Curr Biol 10, |
| | | | | | R260 (2000)) |
| 21 | chaperonin subunit 6a (zeta); chaperonin | 6753324 | 25% | 678 | TRIC component |
| | containing TCP-1 | | | | (Lopez-Fanarraga et |
| | | | | | al., J Struct Biol 135, |
| | | | | | 219 (2001); Leroux et |
| | | | | | al., Curr Biol 10, |
| | | | | | R260 (2000)) |
| 22 | chaperonin subunit 6a (zeta); chaperonin | 6753324 | 27% | 594 | TRIC component |
| | containing TCP-1 | | | | (Lopez-Fanarraga et |
| | | | | | al., J Struct Biol 135, |
| | | | | | 219 (2001); Leroux et |
| | | | | | al., Curr Biol 10, |
| | | | | | R260 (2000)) |

^{A)}numbering according to excised spots from 2D-gel, FIG. 5A.
^{B)}obtained with: MASCOT Matrix science, http://www.matrixscience.com.
^{C)}identified from one-dimensional SDS-PAGE as shown in FIG. 6A (C. Regnard et al., J Cell Sci\ 116, 4181 (2003)).
^{D)}protein of unknown function named nicolin (Backofen et al., Eur J Biochem 269, 5240 (2002)).

### Example II

### Biological Assay for Glutamylase Inhibition

The invention provides a phenotype-based assay for inhibitors of tubulin glutamylation. The assay is a biological assay; that is, it is performed in a host cell, preferably Tetrahymena. It is also referred to herein as an “in vivo” assay or “Assay I.”

Overproduction of the truncated Ttll6Ap-GFP under cadmium-inducible promoter (at 2.5 μg/ml CdCl₂) caused a tight arrest in cell multiplication of Tetrahymena. Decreasing the concentration of cadmium allowed for more growth while increasing it two-fold was lethal within 18 hours. Thus, the extent of growth inhibition by Ttll6Ap is dependent on its intracellular concentration (which in turn is dependent on cadmium concentration). The presence of a compound which inhibits Ttll6Ap activity should partly or completely rescue the cell growth arrest. A preferred embodiment of the in vivo assay is outlined in FIG. 3 (left panel). The assay involves adding CdCl₂ to Tetrahymena cells in which Ttll6Ap expression is controlled by the cadmium-dependent microtubule T1 promoter, distributing cells into wells with compounds, incubating for a time required for uninhibited cells to grow, and reading a fluorescent signal that reflects cell density. Previous studies have established that there is an approximately linear response in the level of protein production using the microtubuleT1 promoter within the range of 0.25-5 μg/ml of CdCl₂ (Shang et al. (2002). Proc Natl Acad Sci 99, 3734-3739). A range of cadmium concentrations is used to create a spectrum of target/inhibitor ratios.

The in vivo assay can be optimized as follows:

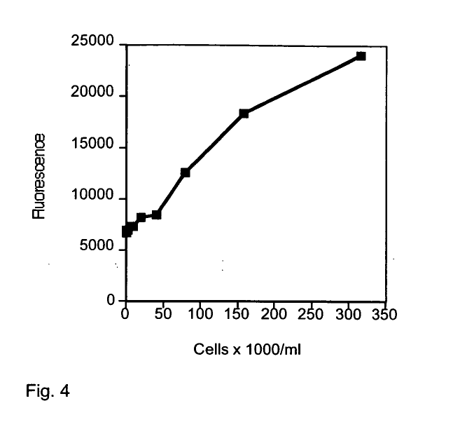

1) A strain with a red fluorescent protein (RFP) reporter is constructed so that cell density can be determined instantly using plate reader (e.g., BMG Fluorostar Optima reader). Fluorescence is beneficial over alternative readout types because there is no need to add detection substrates. In a pilot experiment and without any optimization we were able to detect a signal in living highly mobile GFP-expressing Tetrahymena cells above 10,000 cells/ml (FIG. 4). A simultaneous quantification of RFP and Ttll6Ap-GFP will help in sorting the primary hits. First, a strain that has RFP coding region stably integrated under a strong promoter whose activity is known to reflect cell proliferation (nonessential histone locus HHT1) is constructed. Next, a microtubuleT1-driven Ttll6Ap-GFP transgene is introduced into the nonessential BTU1 locus using a negative selection method (Gaertig et al. (1999) Nat. Biotech. 17, 462-465; U.S. Pat. No. 6,846,481, issued Jan. 25, 2005). As a control, a similar strain containing a version of Ttll6Ap that is inactive due to a mutation in the ATP-binding site (RFP, Ttll6Ap-G422-GFP) is prepared. The simultaneous use of this strain is valuable as a counterscreen for unrelated growth stimulators and compounds that are highly toxic. Our data indicate that Ttll6Ap-overproducing cells upon induction with 2.5 82 g/ml CdCl₂ at density of ˜104 cells/ml divide only once and do not proliferate for at least 12 hrs while controls undergo between 4-5 doublings (Janke et al. (2005) Science 308, 1758-1762 (see Example I)). Thus, the signal to noise ratio could theoretically be ˜20 but the signal could be amplified by RFP fluorescence.

2) Assay conditions can be established for the following parameters:

a) Cadmium concentration and exposure time. An important aspect is the titration of potential inhibitory compounds. As discussed, the presence of a compound at a concentration that depletes the overproduced enzyme should promote cell growth. However, a relevant compound is likely to inhibit cell growth at its higher concentration that also depletes the endogenous glutamylases. This will especially be the case of potential broad specificity inhibitors that affect both TTLL6 and TTLL1 type glutamylases (β-tubulin and α-tubulin preferring, respectively). The issue of titration could be dealt with by using compounds in several different concentrations as it is done routinely in HTS. Alternatively, Ttll6Ap can be induced at several levels by varying the cadmium concentration. This could lower the cost of screened compounds especially if the assay volume can also be reduced (see below). Using 96-well plates, the most optimal range of CdCl₂ concentration to produce the desired signal/noise ratio can be determined. Three concentrations can be chosen: 1) the lowest, 2) mid-range and 3) highest concentration of CdCl₂ that inhibit the multiplication of RFP, Ttll6Ap-GFP cells for a period sufficient to give a signal/noise ratio above 5. If needed, the exposure time can be increased to allow uninhibited cells to produce stronger readout signal.

b) Cell concentration. The lower the initial cell concentration, the lower the background but potentially longer time of incubation may be needed to produce a sufficiently strong signal.

c) Assay volume. The smaller the volume, the smaller the required quantities of screened chemicals. Furthermore, Tetrahymena cells grow faster and to higher densities in smaller drops due to increase aeration, which may increase the signal/noise ratio.

d) DMSO. DMSO is commonly used as a solvent for compounds. Published data indicate that Tetrahymena tolerates well DMSO at up to 2.5% (Nilsson et al. (1974) J Cell Sci 16, 39-47.

e) Assay sensitivity. Assay sensitivity can be evaluated by running assays on plates and including control wells with a series of CdCI₂ concentrations that are progressively lower. Lowering the cadmium concentration will mimic the Ttll6Ap activity-depleting action of an inhibitory compound. The actual effect of lowering CdCl₂ on the level of Ttll6Ap-GFP can be evaluated quantitatively by western blotting (using purified Ttll6Ap as a standard). Thus, we can determine the minimal depletion in the Ttll6Ap activity that can be detected using our assay.

If this assay is used as a primary screen, two secondary screens for hit validation can be used: 1) immunofluorescence and western blotting of compound-treated cells with anti-glutamylation antibodies will determine whether the compound affects the level and pattern of tubulin glutamylation; microscopic observations of treated cells will also determine whether the compound's action gives phenotypic changes that are consistent with deficiency in tubulin, and 2) an in vitro assay with purified enzyme will determine whether the compound directly inhibits the glutamylase enzyme activity. Further assays can be used to determine whether the enzyme is a broad or selective inhibitor and whether it can act on mammalian enzymes.

### Example III

### Cell-Free Assay for Glutamylase Inhibition

The assay is an “in vitro” assay; that is, it is performed in a cell-free environment. The in vitro assay is referred to herein as “Assay II.” An in vitro assay is useful for hit validation (secondary screen) or possibly also as a primary high throughput screen. Assay for the purpose of a secondary screen is described first. The in vivo screen described (Assay I) could identify a large number of compounds. Several mechanisms could give a positive readout: 1) direct inhibition of glutamylases, 2) inhibition of an activator of glutamylases, 3) inhibition of an effector of microtubule glutamylation, 4) stimulation of a tubulin deglutamylase (there is evidence that reverse enzymes exist (Audebert et al. (1993) Mol. Biol. Cell 4, 615-626), 5) inhibition of expression by the MTT1 promoter, 6) growth stimulation by unrelated mechanisms, 7) intrinsic fluorescence of a compound. If we simultaneously read the RFP and GFP signals, we should eliminate compounds which affect the MTT1-driven expression. Compounds that promote growth non-specifically will show an increase in RFP signal in the counterscreen with cells expression inactive glutamylase. Compounds with intrinsic fluorescence will increase signal in the counterscreen as well. Compounds of type 1) (direct inhibitors) can be identified using an in vitro reaction with a purified Ttll6Ap. A reliable in vitro assay has been established for Ttll6Ap using a crude or purified Ttll6Ap, taxol-stabilized brain microtubules, ATP, and tritiated glutamic acid (Janke et al. (2005) Science 308, 1758-1762 (see Example I)). Following the reaction in vitro, products are separated by 1 D SDS-PAGE, blotted onto nitrocellulose and the level of incorporation of tritiated E into α- and β-tubulin is determined for excised bands using liquid scintillation counting. A fairly large number of compounds can be readily screened with a crude cytosolic assay. While this will eliminate some compounds, a relatively pure enzyme will be needed to identify compounds that are direct inhibitors (although compounds that are in categories 2-4 are also of great interest in long-term). Ttll6Ap has been partially purified from cytosol of overproducing cells by a phosphocellulose chromatography and gel filtration (Janke et al. (2005) Science 308, 1758-1762 (see Example I)). An alternative method for purifying the enzyme is possible using a modification of the tandem affinity purification (TAP) method (O'Connor et al. (2005) J Biol Chem 280, 17533-17539, Prathapam et al. (2005) Nat Struct Mol Biol 12, 252-257). The batch-purified enzyme can be used for validation of primary hit compounds using our established (radioactive) assay. The assay is time-consuming (1 day) but highly reliable and suitable as a validation screen.

Because TTLL6Ap is an ATPase, another assay is possible based on the production of ATP-dependent luminescence by luciferase with luciferin substrate. This assay measures the amount of ATP left after the reaction with an ATP consuming-enzyme and the level of luminescence is inversely proportional to the measured enzyme activity. The assay is widely used with kinases and has been used with other ATPases such as myosin (Cheung et al. (2002) Nat Cell Biol 4, 83-88). The assay is shown in in FIG. 3 (right panel). The ATP concentration is first optimized using a standard assay with radiolabelled glutamate. Next, a relatively low ATP concentration (micromolar range) is used, under which the reaction is still robust, to determine the timing of the reaction under which most of ATP is depleted by the activity of the TTLL6Ap. Once these conditions are established, the reaction is performed at 95% of ATP hydrolysis with luciferin and luciferase, and the luminescence signal is measured using the Lmax luminescence plate reader. The reliability and sensitivity of this assay can be determined by running test reactions in the presence of tritiated glutamic acid and analyzing products by both luminescence and liquid scintillation counting. Many parameters of the assay can be optimized, including the enzyme and substrates concentration, pH, Mg2+ concentration. One modification which could substantially lower the cost of high throughput screening is the potential use of ciliary axonemes as a microtubule substrate, in place of standard taxol-stabilized brain microtubules. Tetrahymena microtubules are certainly a formidable substrate in vivo (FIG. 2) and ciliary microtubules can be easily purified at extremely low cost. The assay sensitivity to inhibitors can be determined by using different ratios of ATP and nonhydrolyzable analog, AMP-PNP.

If the in vitro assay (Assay II) is used as a primary screen, the following secondary screens can be used for validation: 1) an in vitro activity assay with purified enzyme and tritiated glutamate; 2) immunofluorescence and western blotting of treated cells with anti-glutamylation antibodies to determine whether the compound affects the level of tubulin glutamylation in vivo (and causes anticipated types of phenotypic changes) which would indicate that it can penetrate the plasma membrane. Finally, using an overexpression in vivo assay (Assay I) we can determine whether the compound inhibition can be relieved by mild overproduction of Ttll6Ap.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A Tetrahymena cell that overexpresses a tubulin glutamylase.

2. A Tetrahymena cell comprising a polynucleotide operably encoding a tubulin glutamylase, wherein the polynucleotide is integrated into a BTU1 locus.

3. The Tetrahymena cell of claim 1 or 2 wherein the polynucleotide comprises an MTT1 promoter region upstream of a region encoding a fusion protein comprising a marker protein and a tubulin glutamylase.

4. The Tetrahymena cell of claim 3 wherein the tubulin glutamylase is a wild-type or a modified tubulin glutamylase.

5. The Tetrahymena cell of claim 3 wherein the tubulin glutamylase comprises Tetrahymena Ttll6Ap glutamylase.

6. The Tetrahymena cell of claim 3 comprising Tetrahymena TTLL6A gene (locus number 25.m00404 at Tetrahymena genome database).

7. The Tetrahymena cell of claim 3 wherein the tubulin glutamylase comprises Tetrahymena Ttll6Ap-Δ₇₁₀ glutamylase.

8. The Tetrahymena cell of claim 3 wherein the marker protein comprises an optically detectable protein.

9. The Tetrahymena cell of claim 3 wherein the marker protein comprises a fluorescent protein

10. A method for identifying an inhibitor of tubulin glutamylase comprising:
contacting the Tetrahymena cell of claim 1 with a candidate inhibitor compound; and
detecting an increase in at least one of cell growth or motility; wherein an increase in cell growth or motility is indicative of tubulin glutamylase inhibition.

11. A method for identifying an inhibitor of tubulin glutamylase comprising:
contacting the Tetrahymena cell of claim 2 with a candidate inhibitor compound; and
detecting an increase in at least one of cell growth or motility; wherein an increase in cell growth or motility is indicative of tubulin glutamylase inhibition.
